(19) United States
(12) Patent Application Publication
(10) Pub. No.: US 2024/0173118 A1
(43) Pub. Date: May 30, 2024

(54) GROMMET WITH COILED REINFORCEMENT FOR USE WITH IN-SITU FENESTRATION

(71) Applicant: Medtronic Vascular, Inc., Santa Rosa, CA (US)

(72) Inventors: David A. GROSSMAN, Santa Rosa, CA (US); Michael A. GLOSS, Santa Rosa, CA (US); Kerry I. PAGE, Santa Rosa, CA (US); Jamie S. DUNAWAY, Santa Rosa, CA (US)

(21) Appl. No.: 18/384,414

(22) Filed: Oct. 27, 2023

Related U.S. Application Data
(60) Provisional application No. 63/428,998, filed on Nov. 30, 2022.

(51) Int. Cl. A61F 2/06; A61F 2/07; A61F 2/966

(52) U.S. Cl. CPC A61F 2/064; A61F 2/07; A61F 2/966; A61F 2002/061; A61F 2002/067; A61F 2220/0033; A61F 2230/0091

(57) ABSTRACT

An endovascular grommet includes a central member including a central portion and first and second portions extending from the central portion. The endovascular grommet further includes an inner ring connected to the central member, a first outer ring connected to the central member, a second outer ring connected to the central member, a first coil secured to the first portion of the central member, and a second coil secured to the second portion of the central member. The grommet is configured for deployment within an in-situ fenestration.

### CROSS-REFERENCE TO RELATED APPLICATION

This application claims the benefit of U.S. provisional application Ser. No. 63/428,998 filed on Nov. 30, 2022, the disclosure of which is hereby incorporated in its entirety by reference.

### TECHNICAL FIELD

The present disclosure relates to grommets with coiled reinforcements for use with in-situ fenestrations.

### BACKGROUND

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

### SUMMARY

In one embodiment, an endovascular grommet is disclosed. The endovascular grommet includes a central member including a central portion and first and second portions extending from the central portion. The endovascular grommet further includes an inner ring connected to the central member, a first outer ring connected to the central member, a second outer ring connected to the central member, a first coil secured to the first portion of the central member, and a second coil secured to the second portion of the central member. The grommet is configured for deployment within an in-situ fenestration.

In another embodiment, an endovascular stent graft system is disclosed. The endovascular stent graft system includes a main stent graft defining a fenestration and including stent graft material. The endovascular stent graft system also includes a peripheral stent graft. The endovascular stent graft also includes a grommet forming an interface between the main stent graft and the peripheral stent graft to resist leakage through the fenestration. The grommet includes a central member including a central portion and first and second portions extending from the central portion, an inner ring connected to the central member, a first outer ring connected to the central member, a second outer ring connected to the central member, a first coil secured to the first portion of the central member, and a second coil secured to the second portion of the central member. The peripheral stent graft extends through the main stent graft and the grommet.

In yet another embodiment, an endovascular grommet deployment system is disclosed. The endovascular grommet deployment system includes a capsule and a grommet. The grommet has a constrained position within the capsule and a deployed position outside of the capsule. The grommet includes a central member, an inner ring connected to the central member, first and second rings connected to the central member, and first and second coils secured to the central member. The grommet is configured to straddle stent graft material defining a fenestration when the grommet is in the deployed position.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIG. 2A depicts a partial cut away, schematic, side view of an abdominal aorta and right and left renal arteries extending therefrom when a main stent graft extends an aneurysm of the abdominal aorta, a peripheral stent extends from the stent graft into the right renal artery, and a gasket configured to create an interface between the peripheral stent and the aneurysm.

FIG. 2B depicts a cut away, schematic, side view of the peripheral stent and the gasket forming an interface.

FIG. 2C depicts an isolated, perspective view of the gasket including an inner ring, first and second outer rings, and first and second outer rings.

FIG. 2D depicts an isolated side view of the gasket where the first and second outer rings are configured to overlap and/or nest to pinch and/or grip the main stent graft.

FIG. 3A depicts an isolated, schematic view of the first and second outer rings of the gasket.

FIG. 3B depicts an isolated, schematic view of a center member formed of a first material.

FIG. 3C depicts an isolated, schematic view of a center member of a second material.

FIG. 3D depicts an isolated, schematic view of the inner ring, and first and second outer rings.

FIGS. 4A, 4B, and 4C depict partial cut away, schematic views of the gasket of FIG. 2A depicting the internal forces within the gasket.

FIGS. 5A, 5B, 5C, and 5D depict schematic side views of steps of a deployment system to deploy the gasket of FIG. 2A within a fenestration made in a stent graft material.

FIG. 6A depicts a schematic, perspective, side view of a gasket tracked through a fenestration in a stent graft material of a stent graft and extending into a peripheral artery.

FIGS. 6B and 6C depict a schematic, fragmented, perspective view and a schematic, fragment side view, respectively, of the gasket in a partially deployed position.

FIGS. 6D and 6E depict a schematic, fragmented, perspective view and a schematic, fragmented side view, respectively, of a retainer configured to retain a first coil of the grommet in a constrained position and to release the first coil into an expanded position.

FIG. 6F depicts a schematic, perspective view of a stent graft including the gasket in a fully deployed position with a fenestration.

FIG. 6G depicts a schematic, perspective view of a stent graft with the gasket in a fully deployed position within the fenestration and peripheral stent extending through the gasket and the fenestration into a peripheral artery.

FIG. 7A depicts a schematic, side view of a grommet delivery system extending through a stent graft deployed within an aneurysm of an abdominal aorta with right and left renal arteries extending therefrom.

FIG. 7B depicts a schematic, side view of the grommet delivery system in which delivery device partially extends through a fenestration and into the left renal artery.

FIG. 7C depicts a schematic, side view of the grommet delivery system during deployment of a grommet into the fenestration in the stent graft material in the stent graft.

FIG. 8A depicts a perspective view of a grommet configured to be delivered to and deployed at a fenestration with a grommet delivery system.

FIG. 8B depicts a schematic, perspective view of the grommet where second lobe on the right side is in a collapsed state (e.g., during delivery of the grommet to a deployment site).

FIG. 8C depicts a schematic, partial cross-sectional, side view of the grommet loaded into the grommet delivery device extending from the distal end of the grommet delivery catheter (e.g., a pathway catheter).

FIG. 8D depicts a schematic, partial cross-sectional, side view of the grommet where the second arms are released from a shuttle cap to spring into a deployed position.

FIGS. 9A and 9B depict a schematic, side view and a schematic, perspective view, respectively, of a grommet including a skirt and first and second frame members attached to the skirt at the first and second ends thereof.

FIG. 9C depicts a schematic, perspective view of the first and second frame members and the skirt.

FIG. 9D depicts a schematic, perspective view of the skirt.

FIGS. 10A, 10B, 10C, and 10D depict schematic side views of operations for creating an in-situ fenestration and deploying a grommet within a fenestration according to one or more embodiments.

FIG. 11A depicts a schematic, perspective view of a procedure for loading a grommet into a capsule of a delivery system.

FIG. 11B depicts a schematic, perspective view of a procedure for deploying the grommet into a fenestration.

FIG. 12 depicts a schematic, side view of a grommet deployed between a peripheral artery and an aortic artery.

FIG. 13A depicts a schematic, side view of a branching artery branching from an aortic artery with a stent graft deployed within the aortic artery and a grommet deployed within the in-situ fenestration.

FIG. 13B depicts a schematic, fragmented, perspective view of the stent graft having a fenestration with the grommet deployed therein.

FIG. 13C depicts a schematic, perspective, isolated view of the frame of the grommet.

FIG. 13D depicts a schematic, perspective view of the frame and the skirt of the grommet.

FIG. 14A depicts a schematic, side view of the grommet being delivered within a capsule.

FIG. 14B depicts a schematic, side view of the grommet during a deployment operation.

FIGS. 15A, 15B, 15C, and 15D depict schematic side views of operations for creating an in-situ fenestration and deploying a grommet within the fenestration according to one or more embodiments.

FIGS. 16A, 16B, 16C, 16D, and 16E depict schematic side and perspective views of a procedure for deploying a grommet into a fenestration.

FIG. 17A depicts a schematic, side view of a grommet delivery device including a shaft according to one embodiment.

FIG. 17B depicts a schematic, cross-sectional view of the inner shaft according to one embodiment.

FIG. 17C depicts a schematic, side view of an adhesive port situated between the inner shaft and an outer shaft.

FIG. 17D depicts a schematic, side view of the grommet delivery device in a second deployment position.

FIG. 18A depicts a schematic, perspective view of a grommet in an expanded state.

FIG. 18B depicts a schematic, side view of the grommet in the expanded state.

FIG. 18C depicts a schematic, plan view of a grommet in a crimped state.

FIG. 18D depicts a schematic, perspective view of the grommet in the crimped state.

FIGS. 19A, 19B, 19C, 19D, 19E, and 19F depict schematic, side views of an aortic arch branching into a brachiocephalic artery, a left common carotid artery, and a left subclavian artery with a stent graft deployed in the aortic arch and a delivery catheter tracking transfemorally to the stent graft.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

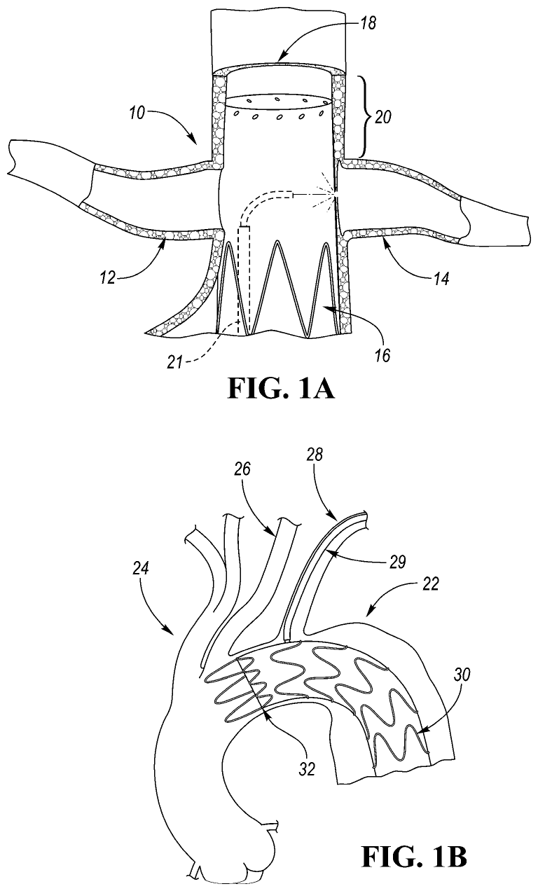

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching cannulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Eye cautery (e.g., thermal energy) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel cannulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one embodiment, a grommet (e.g., gasket) configured to form an interface between a stent graft and a peripheral stent/stent graft is disclosed. The gasket may include a central member connected to an inner ring and first and second outer rings. The gasket may further include first and second coils connected to an outer peripheral region of the central member. The central member may include first and second portions extending from a central portion of the central member. The first coil may be secured to the first portion of the central member. The second coil may be secured to the second portion of the central member. The gasket may be deployed within an in-situ fenestration to resist leakage therethrough and/or tearing of the in-situ fenestration.

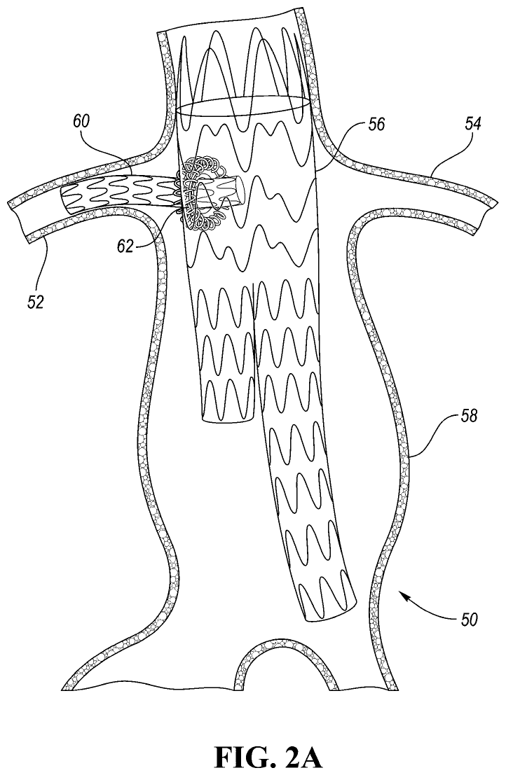

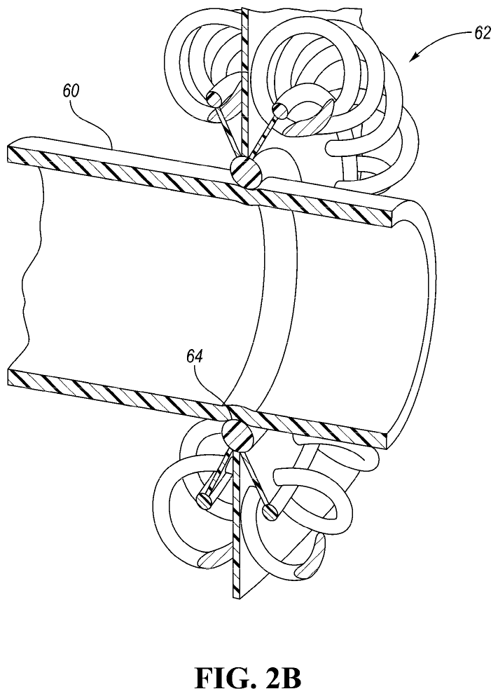

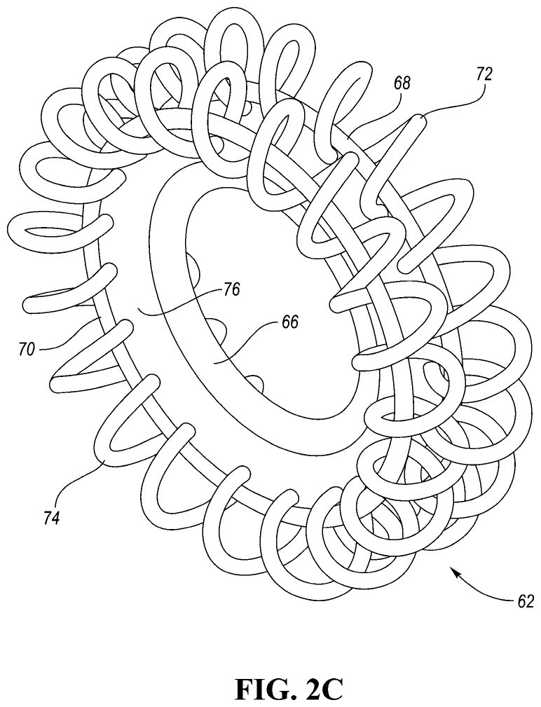

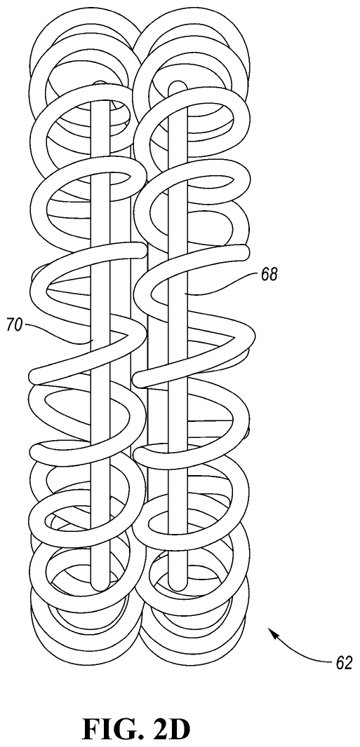

FIG. 2A depicts a partial cut away, schematic, side view of abdominal aorta 50 and right and left renal arteries 52 and 54 extending therefrom where main stent graft 56 extends within aneurysm 58 of abdominal aorta 50, peripheral stent graft 60 extends from stent graft 56 into right renal artery 52, and gasket 62 configured to create an interface between peripheral stent graft 60 and main stent graft 56. FIG. 2B depicts a cut away, schematic, side view of peripheral stent graft 60 and gasket 62 forming interface 64. FIG. 2C depicts an isolated, perspective view of gasket 62 including inner ring 66, first and second outer rings 68 and 70, and first and second outer coils 72 and 74. Inner ring 66 is configured to seal against the outer surface of peripheral stent graft 60. Center member 76 extends between inner ring 66 and first and second outer rings 68 and 70. FIG. 2D depicts an isolated side view of gasket 62 where first and second outer rings 68 and 70 are configured to overlap and/or nest to pinch and/or grip main stent graft 56.

In one or more embodiments, gasket 62 is configured to reinforce an in-situ fenestration formed in main stent graft 56 to create interface 64 between peripheral stent graft 60 and main stent graft 56. In one or more embodiments, gasket 62 is configured to allow for a relatively wide range of fenestration methods and resulting fenestration edge conditions. In one or more embodiments, gasket 62 is configured to interface between peripheral stent graft 60 and main stent graft 56 to provide a seal between both grafts 56 and 60 and aneurysm 58. The seal may provide the capability to select different in-situ fenestration locations that may not be aligned with a peripheral ostium. Gasket 62 may absorb any forces exerted by a peripheral stent misaligned with a peripheral ostium. Gasket 62 may increase the effectiveness of mechanical fenestration devices. Gasket 62 may produce minimal or no fenestration debris (e.g., a cutting tool may be used to create a relatively simple fenestration slit as opposed to a hole that may have removed material for externalizing).

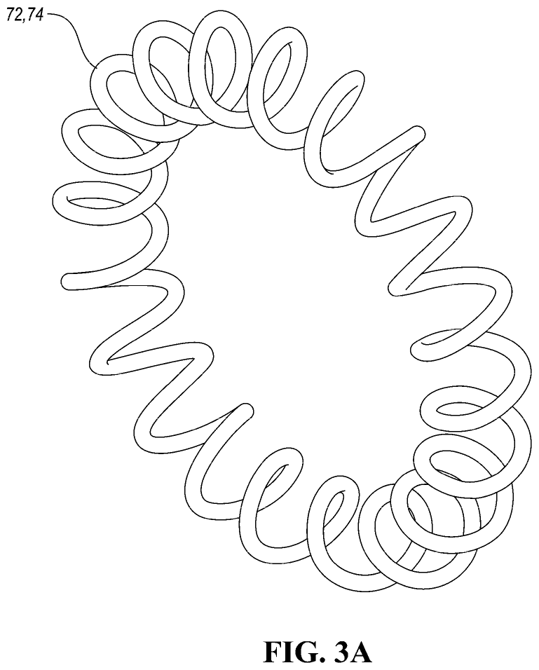

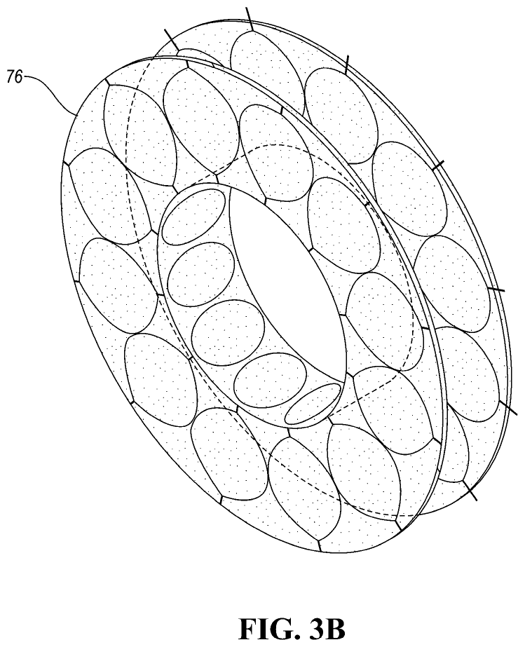

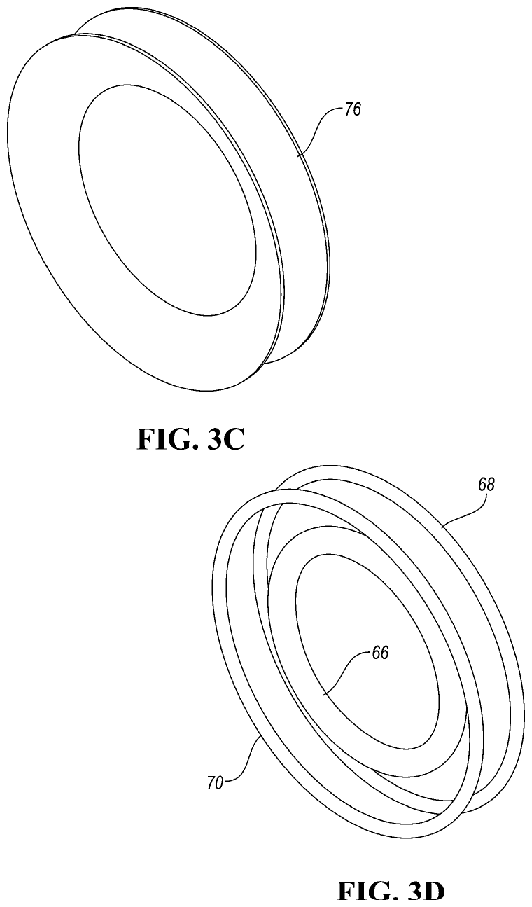

FIG. 3A depicts an isolated, schematic view of one of the first and second outer coils 72 and 74 of gasket 62. First and/or second outer coils 72 and/or 74 may be formed of a shape memory material, such as Nitinol. First and/or second outer coils 72 and/or 74 may be formed from a straight wire into a bent, coil shape as it is secured to center member 76. First and/or second outer coils 72 and/or 74 may be coated with a coating to resist the likelihood of fretting fractures in the coil and stent graft sinusoids. The coating material may include polytetrafluoroethylene (PTFE). First and/or second outer coils 72 and/or 74 may have a helical or coiled shape.

FIG. 3B depicts an isolated, schematic view of center member 76 formed of a first material. FIG. 3C depicts an isolated, schematic view of center member 76 formed of a second material. As shown in FIGS. 3B and 3C, center member 76 has a dual opposing conical frustrum shape. In one or more embodiments, inner ring 66, first and second outer rings 68 and 70, and first and second coils 72 and 74 are connected to center member 76. These connections may apply tension to center member 76 through first and/or second coils 72 and/or 74. One or more of the connections may be formed of sutures or other filaments. First and/or second coils 72 and/or 74 may be threaded through the first and/or second conical frustums of center member 76. Alternatively, the connections may be formed via adhesive or welding. As shown in FIG. 3B, the first material is a mesh structure covered with a polymeric material. The mesh structure may be made of a metal material and the polymeric material may be PTFE. As shown in FIG. 3C, the second material is a fabric material.

FIG. 3D depicts an isolated, schematic view of inner ring 66, and first and second outer rings 68 and 70. Inner ring 66, first outer ring 68, and/or second outer ring 70 may be configured to reinforce center member 76. One or more of the rings may be formed of a collapsible, malleable, fuzzy, tacky, and/or squishy material configured to promote sealing. One or more of the rings may also be collapsible so that the rings may fit into a delivery system. The material of one or more of the rings may be integrated with a hydrogel and/or biologic configured to promote expansion and/or ingrowth in the presence of blood. The inner sealing ring diameter may be used to define peripheral stent graft size compatibility.

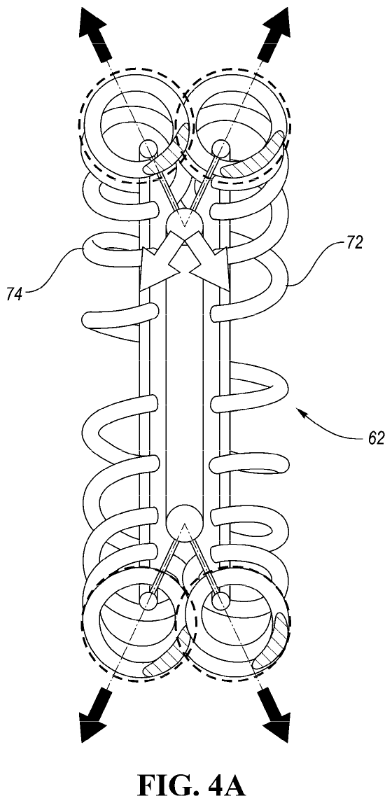

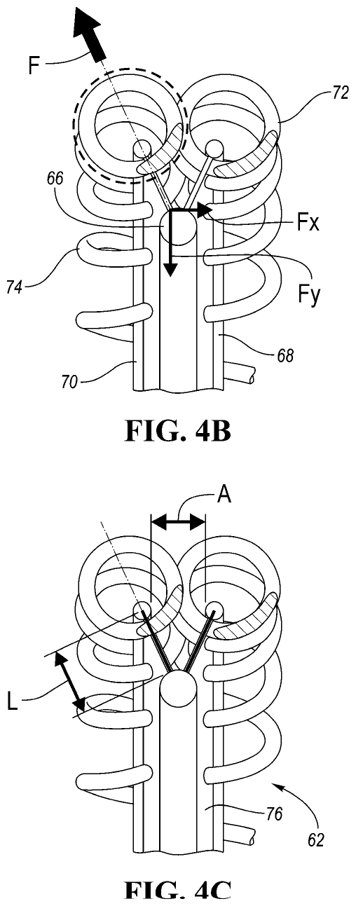

FIGS. 4A, 4B, and 4C depict partial cut away, schematic views of gasket 62 depicting the internal forces within gasket 62. As shown by the outward arrows and cross sections from first and second outer coils 72 and 74 on FIG. 4A, first and second outer coils 72 and 74 exert an outward force on gasket 62. These outward forces are balanced by an inward force exerted by inner ring 66 as shown by the inward arrows on FIG. 4A. As shown in FIG. 4B, the inward resistive force (F_{y}) exerted by inner ring 66 balances with the spring outward force (F) of first and second outer coils 72 and 74. First and second outer rings 68 and 70 are configured to exert a pinching force (Fₓ) configured to pinch stent material around an in-situ fenestration. In one or more embodiments, the pinching force is driven by the shape of center member 76 and other design features of gasket 62. The pinching force may be adjusted by changing one or more of the following design parameters: coil radial outward force (F), fabric cone length (L), and cone angle (A). Fabric cone length (L) and cone angle (A) are shown on FIG. 4C.

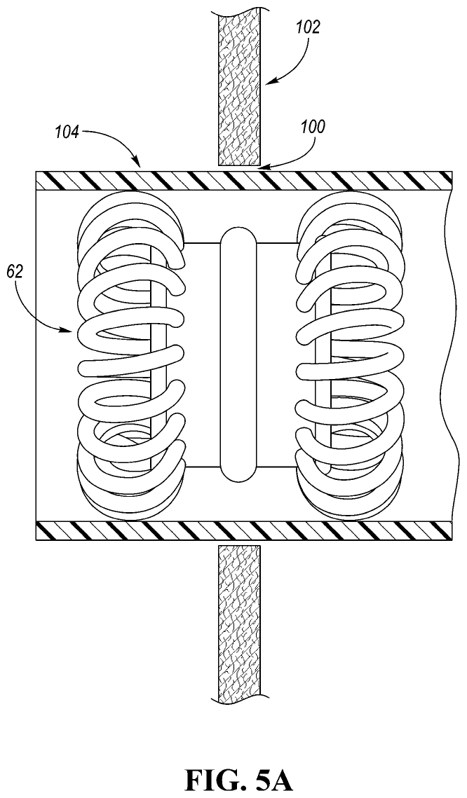

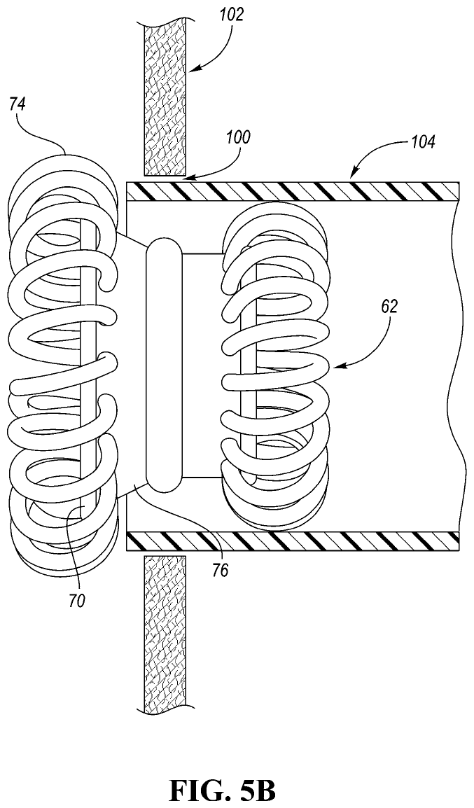

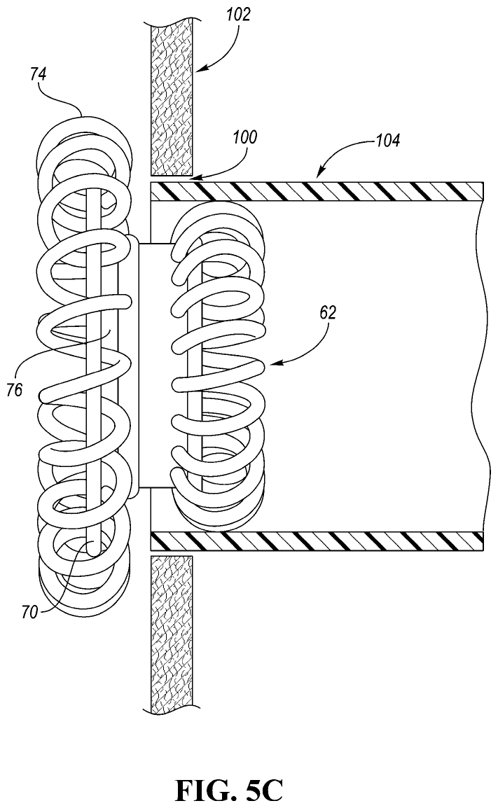

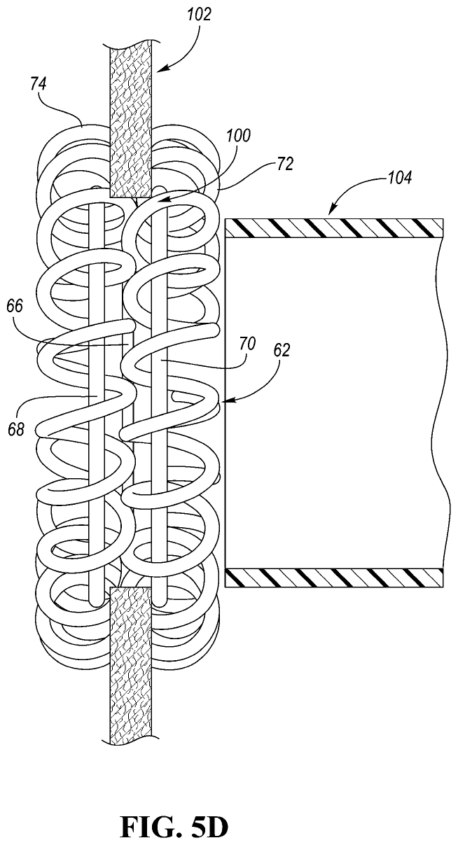

FIGS. 5A, 5B, 5C, and 5D depict schematic side views of steps of deployment system to deploy gasket 62 within fenestration 100 made in stent graft material 102. In FIG. 5A, gasket 62 is delivered to fenestration 100 within capsule or sheath 104. Capsule 104 may be made of a rigid material configured to hold gasket 62 in a constrained position within capsule 104 during delivery to fenestration 100. As shown in FIG. 5B, gasket 62 is partially deployed within fenestration 100 by either retracting capsule 104 or advancing gasket 62 such that second coil 74, second outer ring 70, and a portion of central member 76 radially expand. Second coil 74 may be retained with an inner tension member configured to allow for controlled release and recapture of gasket 62. As shown in FIG. 5C, second coil 74, second outer ring 70, and a portion of central member 76 expand further in a radially direction. Capsule 104 is configured to transfer axial force through gasket 62 for deployment. In FIG. 5D, gasket 62 is fully deployed within fenestration 100 by further retracting capsule 104 or advancing gasket 62 such that first and second coils 72 and 74, first and second outer rings 68 and 70, inner ring 66, and central member 76 are radially expanded. As shown in FIG. 5D, gasket 62 pinches stent graft material 102 between first and second coils 72 and 74, and first and second outer rings 68 and 70. In one or more embodiments, in the fully deployed position, inner ring 66 is aligned with stent graft material 102 to provide strength to the fitting between gasket 62 and stent graft material 102.

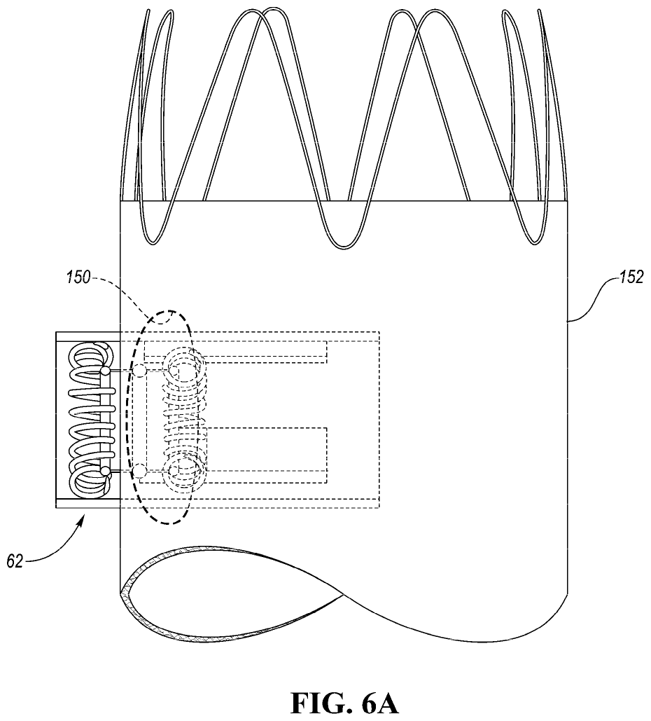

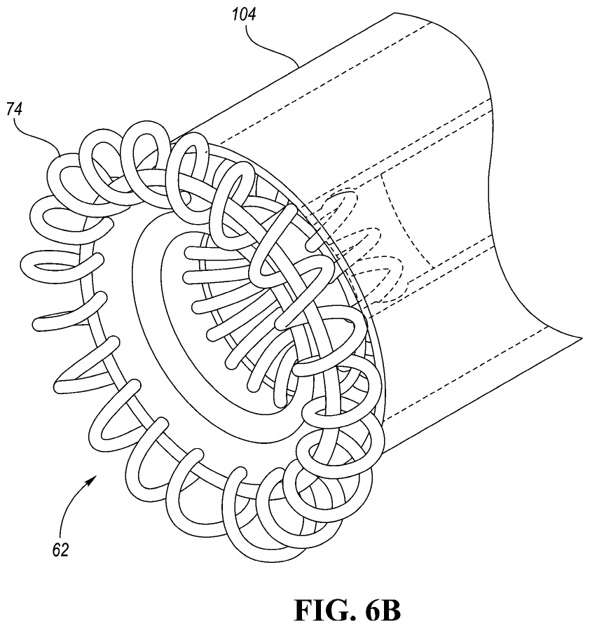

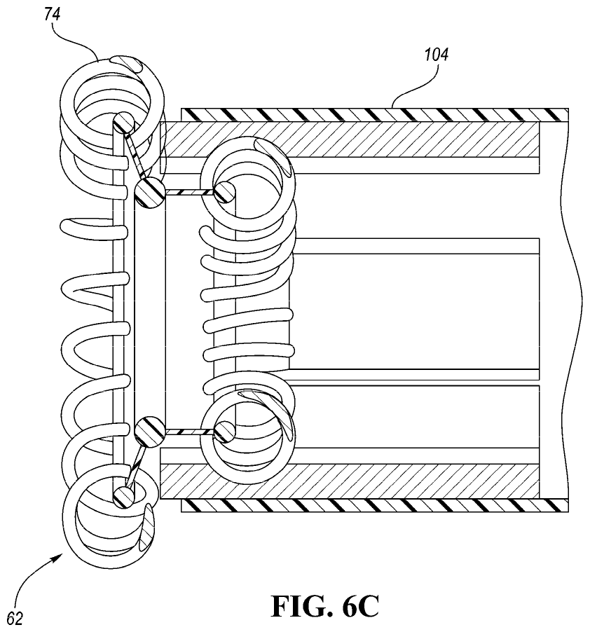

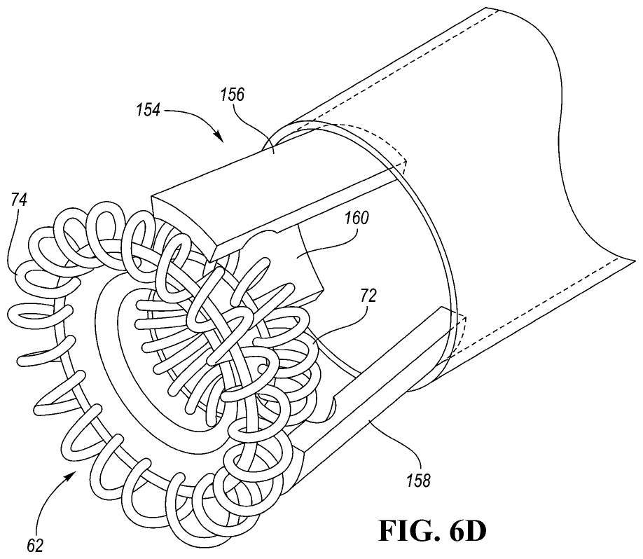

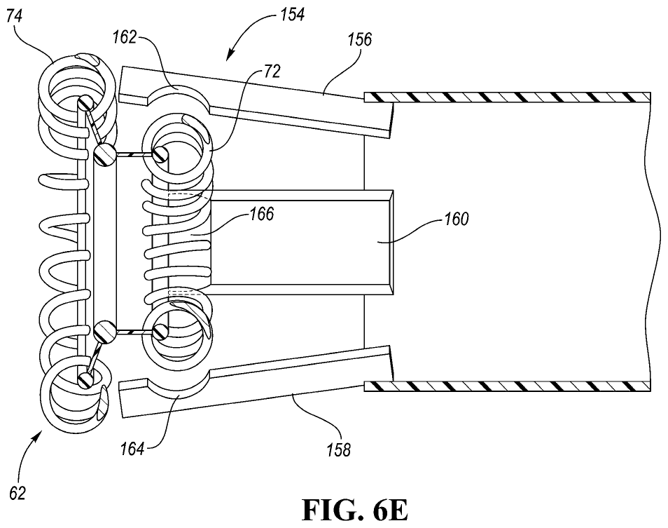

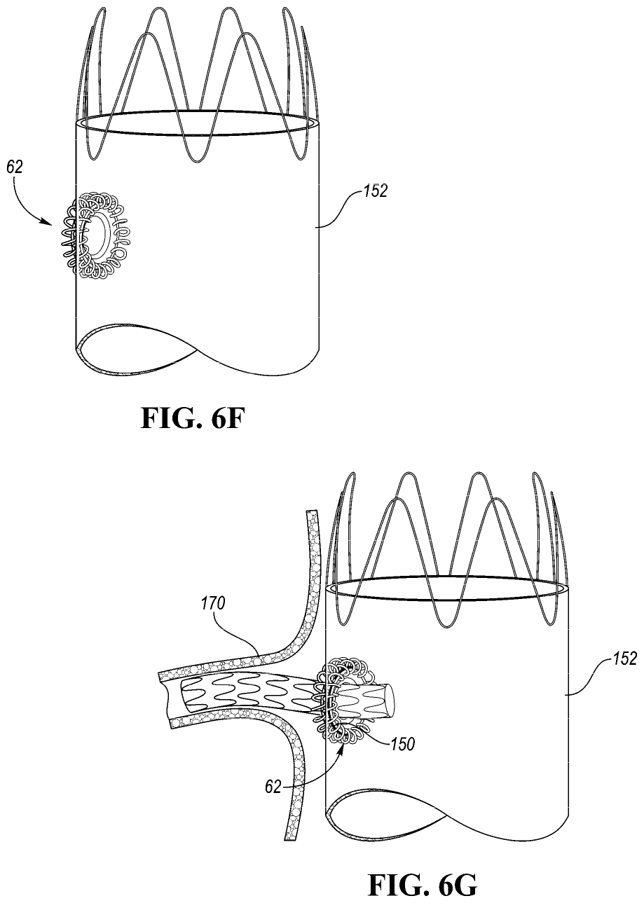

FIG. 6A depicts a schematic, perspective, side view of gasket 62 tracked through fenestration 150 in stent graft material of stent graft 152 and extending into a peripheral artery. The peripheral artery is located with a guidewire and then stent graft 152 is deployed. After fenestration 150 is created at a desired location relative to the peripheral artery, gasket 62 is tracked through fenestration 150.

FIGS. 6B and 6C depict a schematic, fragmented, perspective view and a schematic, fragmented side view, respectively, of gasket 62 in a partially deployed position. In FIGS. 6B and 6C, capsule 104 is retracted relative to gasket 62 to expand second coil 74 beyond the distal end of capsule 104. Second coil 74 is expanded on the outside of stent graft 152.

FIGS. 6D and 6E depict a schematic, fragmented, perspective view and a schematic, fragmented side view, respectively, of retainer 154 configured to retain first coil 72 in a constrained position and to release first coil 72 into an expanded position. Retainer 154 may be formed of a flexible material (e.g., a flexible metal material). In one or more embodiments, a pulling force may be exerted on gasket 62 through a pulling member to determine the position of second coil 74. If the pulling member freely retracts, then a determination may be made that gasket 62 is misaligned (e.g., gasket 62 did not track through fenestration 150). Upon determining a misalignment, capsule 104 may be advanced to recapture second coil 74. As shown in FIGS. 6D and 6E, retainer 154 includes first, second, and third prongs 156, 158, and 160. Each of the first, second, and third prongs 156, 158, and 160 include first, second, and third inwardly curved channels 162, 164, and 166, respectively, following the profile of the outside of first coil 72. First, second, and third inwardly curved channels 162, 164, and 166 are configured to collectively retain first coil 72 in a constrained position during delivery and during deployment of second coil 74. Upon determining that second coil 74 is aligned on the outside of stent graft 152, capsule 104 may be retracted further to outwardly release first, second, and third prongs 156, 158, and 160 from first coil 72 such that first coil 72 expands on the inside of stent graft 152. FIG. 6F depicts a schematic, perspective view of stent graft 152 including gasket 62 in a fully deployed position within fenestration 150.

FIG. 6G depicts a schematic, perspective view of stent graft 152 with gasket 62 fully deployed within fenestration 150 and peripheral stent graft 168 extending through gasket 62 and fenestration 150 into peripheral artery 170. As shown in FIG. 6G, capsule 104 has been retracted from the delivery site. Once capsule 104 has been retracted, peripheral stent graft 168 may be tracked through stent graft 152 and gasket 62 and into peripheral artery 170 and thereafter deployed.

In one or more embodiments, gasket 62 is configured to provide a flexible dock to deploy a peripheral stent into an in-situ aortic stent graft fenestration. Gasket 62 may be configured to not impact the design of the stent graft and/or peripheral stent. In one or more embodiments, gasket 62 is configured to resist fenestration hole tearing. In one or more embodiments, gasket 62 is configured to resist leakage from the stent graft through the in-situ fenestration hole.

In one or more embodiments, a grommet configured to form an interface between a stent graft and a peripheral stent or stent graft is disclosed. The grommet may include a body and torsion springs and first and second arms extending from the torsion springs. The first and second arms are connected to the body. The torsion springs may be configured to exert a torque and/or rotary force on the first and second arms to translate arms from a first position to a second position. The second position may be a natural position whereas the first position may be a position achieved when an external force is applied to the first and/or second arms. The first position may be a collapsed position where a grommet delivery device exerts an external force and the second position may be a deployed position after the torsion springs have exerted a force to overcome the external force (or the external force is no longer being exerted on the first and/or second arms). The grommet may be deployed within an in-situ fenestration to resist leakage therethrough and/or tearing of the in-situ fenestration.

In one or more embodiments, a device configured to connect a grommet to an in-situ fenestration is disclosed. The grommet may be configured to cover the periphery of the in-situ fenestration with a spring-loaded frame while maintaining the opening formed by the in-situ fenestration. The grommet may be installed in a spring-loaded state on a grommet delivery catheter. One side of the grommet may collapse and be held in the collapsed state by a grommet delivery catheter. The grommet delivery catheter is inserted into the patient's vasculature and is aligned with the fenestration and the branch opening. A distal region of the catheter is advanced into the branch opening. A balloon connected to the grommet delivery device is configured to push forward a cap on the grommet delivery device to release the arms/lobes which deploys the grommet via the release of the collapsed side of the grommet. In one or more embodiments, the grommet springs into place over the fenestration, thereby covering the fibers with a spring-loaded frame to maintain the opening of the in-situ fenestration. At this point in the process, the grommet deliver catheter may be removed from the patient's vasculature.

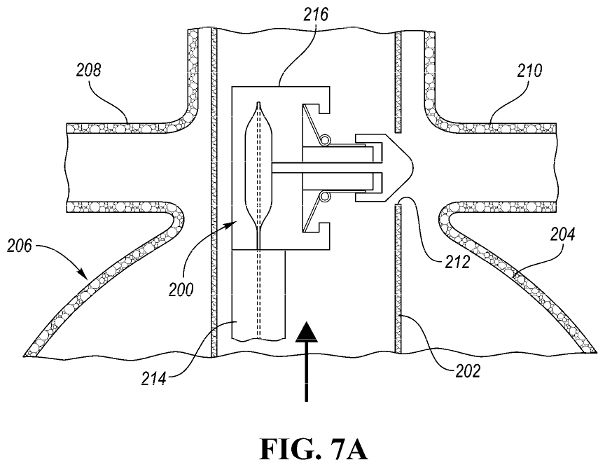

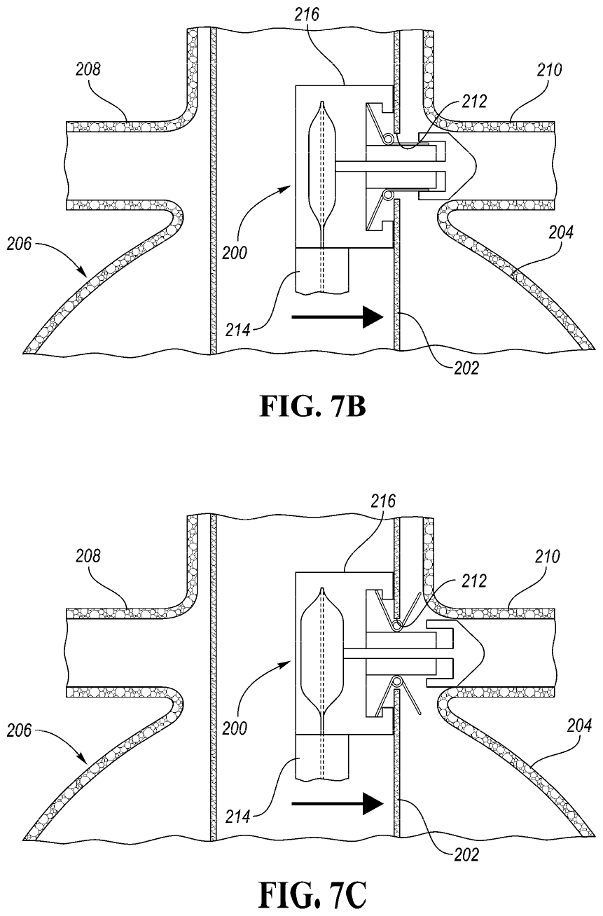

FIG. 7A depicts a schematic, side view of grommet delivery system 200 extending through stent graft 202 deployed within aneurysm 204 of abdominal aorta 206 with right and left renal arteries 208 and 210 extending therefrom. As shown in FIG. 7A, in-situ fenestration 212 is formed in the stent graft material of stent graft 202. Fenestration 212 is aligned with left renal artery 210, although it may be formed to align with other peripheral arteries covered by stent graft 202 (e.g., right renal artery 208). Grommet delivery system 200 includes catheter 214 and delivery device 216 configured to track through catheter 214. As shown in FIG. 7A, delivery device 216 extends beyond the distal end of catheter 214 to align with fenestration 212 and the left renal artery 210.

FIG. 7B depicts a schematic, side view of grommet delivery system 200 in which delivery device 216 partially extends through fenestration 212 and into left renal artery 210. As shown in FIG. 7B, delivery device 216 moves in an axial direction to partially track through fenestration 212 as is described in further detail herein.

FIG. 7C depicts a schematic, side view of grommet delivery system 200 during deployment of a grommet into fenestration 212 in the stent graft material in stent graft 202. In one or more embodiments, and as further described herein, a mechanism may be actuated by a balloon that releases one side of a folding grommet from grommet delivery system 200. The folding grommet may spring into place over the periphery of fenestration 212, thereby covering the fibers of the stent graft material with a spring-loaded frame and fabric structure that is configured to maintain the opening of fenestration 212.

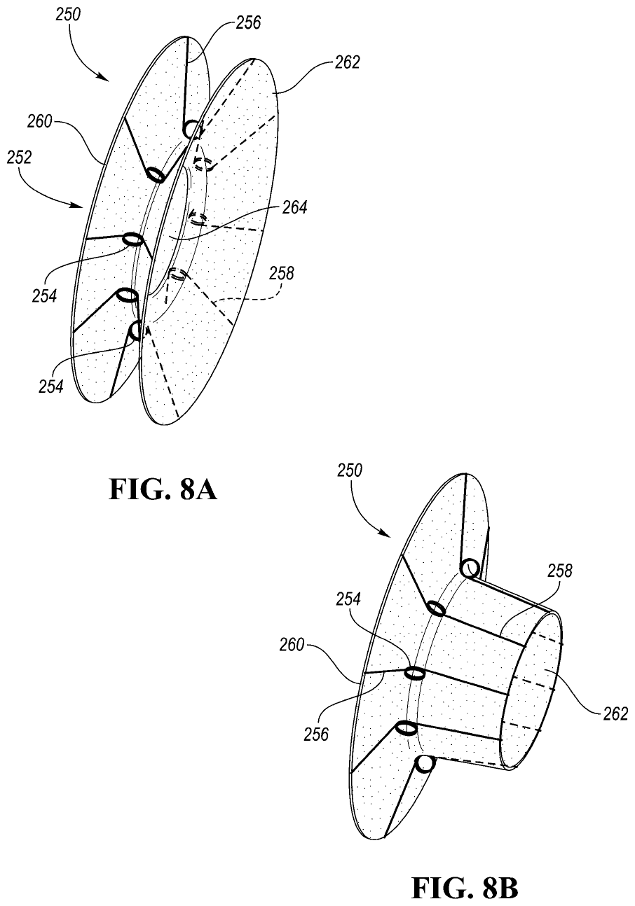

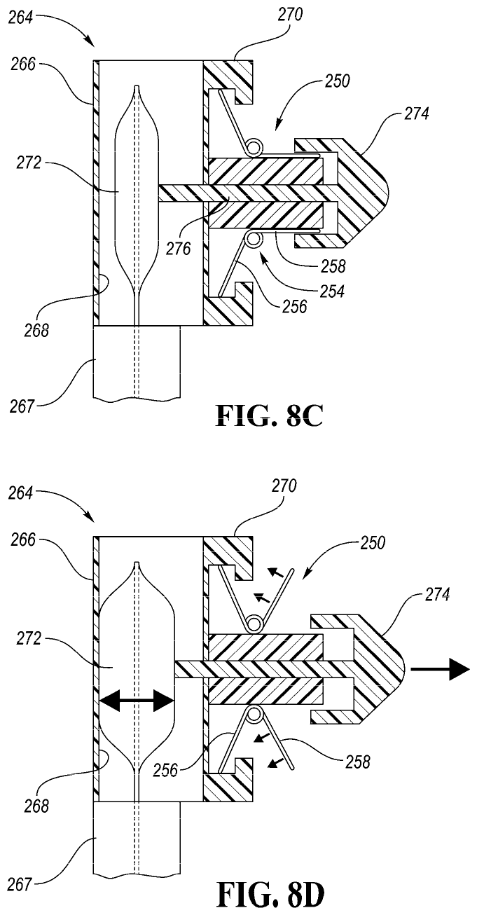

FIG. 8A depicts a perspective view of grommet 250 configured to be delivered to and deployed at fenestration 212 with grommet delivery system 200. Grommet 250 includes body 252 and torsion springs 254 with first and second arms 256 and 258 extending therefrom. Torsion springs 254 with first and second arms 256 and 258 radially spaced around the circumference of body 252. Body 252 may be formed of a fabric material and first and second arms 256 and 258 may be sewn into the fabric material. Torsion springs 254 may also be sewn into the fabric material of body 252. Body 252 includes first and second frustoconical lobes 260 and 262 joining at middle section 264. First arms 256 extend into first frustoconical lobes 260 and second arms 258 extend into second frustoconical lobes 262. Torsion springs 254 may contact middle section 264 of body 252. Middle section 264 may be configured to sew the torsion springs within the fabric body to control how much the diameter can contract during deployment to allow a release mechanism from the retaining lip.

As shown in FIG. 8A, grommet 250 includes 10 torsion springs and arm sets, but the number of torsion springs and arm sets may less or more (e.g., 4, 5, 6, 7, 8, 9, 11, 12, 13, and 14) depending on the implementation. As shown in FIG. 8A, torsion springs 254 and first and second arms 256 and 258 are equally spaced around the circumference of body 252, although in alternative embodiments the torsion springs 254 and first and second arms 256 and 258 may be unequally spaced around the circumference.

Grommet 250 is shown in a deployed state in FIG. 8A where first and second lobes 260 and 262 are configured to contact the outside and inside surfaces of a stent graft. Lobes 260 and/or 262 may be collapsed into a collapsed state for delivery through a catheter of a grommet delivery system as described herein.

FIG. 8B depicts a schematic, perspective view of grommet 250 where second lobe 262 on the right side is in a collapsed state (e.g., during delivery of grommet 250 to a deployment site). In the collapsed state, second lobe 262 has a conical shape tapering inward away from first lobe 260. As shown in FIG. 8B, torsion springs 254 are helical springs configured to exert a torque and/or rotary force on first and second arms 256 and 258 to translate arms from a first position to a second position. The second position may be a natural position whereas the first position may be a position achieved when an external force is applied to first and/or second arms 256 and/or 258. In one or more embodiments, the first position may be the collapsed position where the grommet delivery device exerts the external force and the second position may be a deployed position after torsion springs 254 have exerted a force to overcome the external force (or the external force is no longer being exerted on first and/or second arms 256 and/or 258).

FIG. 8C depicts a schematic, partial cross-sectional, side view of grommet 250 loaded into grommet delivery device 264 extending from the distal end of grommet delivery catheter 267 (e.g., a pathway catheter). As shown in FIG. 8C, grommet delivery device 264 includes base portion 266 forming base cavity 268, retention lip 270 extending from base portion 266, balloon 272 housed within base cavity 268, shuttle cap 274, and shuttle connector 276 attaching balloon 272 to shuttle cap 274. Balloon 272 is shown in a deflated state in FIG. 8C. Retaining lip 270 is configured to hold first arms 256 in an open position (e.g., a partially deployed position). Shuttle cap 274 is configured to hold second arms 258 in a collapsed position. Shuttle cap 274 is configured to advance and/or retract to deploy second arms 258 of grommet 250.

FIG. 8D depicts a schematic, cut away, side view of grommet 250 where second arms 258 are released from shuttle cap 274 to spring into a deployed position. When balloon 272 expands from the deflated state (shown in FIG. 8C) into the inflated state (shown in FIG. 8D), balloon 272 pushes shuttle connector linearly outward (e.g., perpendicular to the longitudinal axis of the delivery catheter 267), thereby releasing second arms 258 from shuttle cap 274 such that second arms 258 translate from the collapsed position into the deployed position. Balloon 272 may be expanded (e.g., inflated) using a balloon inflation mechanism (e.g., a pump). The force created by second arms 258 translating into the deployed position draws first arms 256 out of retaining lip 270 and into the deployed position. For instance, the inner diameter of the grommet reduces when arms 256 and 258 are in the deployed position, allowing firm arms 256 to escape retaining lip 270. At this point in the deployment process, first and second arms 256 and 258 are both in the deployed position, thereby gripping the stent graft material therebetween and protecting the fibers thereof by encapsulating them. Grommet 250 is completely detached from grommet delivery device 264 such that grommet delivery device 264 and grommet delivery catheter 267 can be removed from the patient's vasculature.

One or more embodiments have one or more of the following benefits. The grommet deployment procedure can be performed in-situ. The deployed grommet captures frayed fabric ends in a controlled manner. The grommets may be delivered in various sized used to create variable hole sizes.

The grommet delivery system may utilize a steerable catheter or a guide wire. While grommet delivery system 200 includes delivery device 216 deploying a grommet deployed perpendicular to catheter 214 (e.g., side face deployment), in other embodiments, a steerable catheter or other catheter may be used such that the grommet is deployed axially relative to the catheter axis (e.g., end deployment).

In one or more embodiments, a grommet with expansion members is disclosed. The grommet includes a skirt and first and second frame members attached to the skirt. The skirt may include first and second outer portions connected to each other through a middle portion. Middle portion defines a channel to permit blood flow therethrough. The grommet may be deployed within an in-situ fenestration to resist leakage therethrough and/or tearing of the in-situ fenestration.

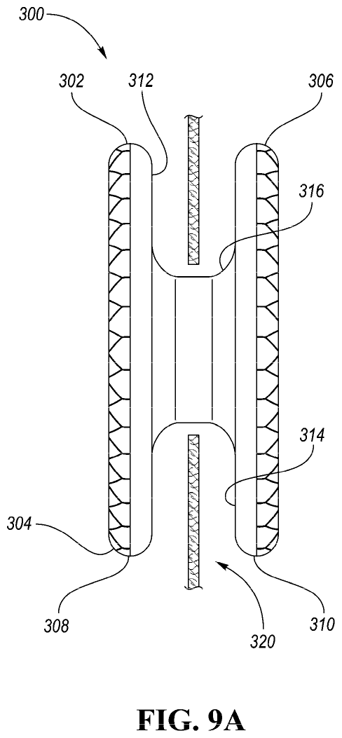

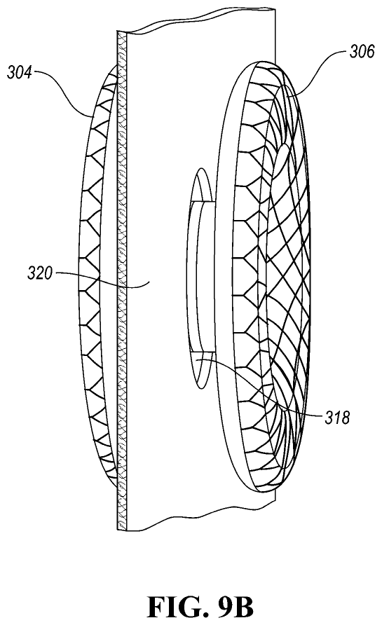

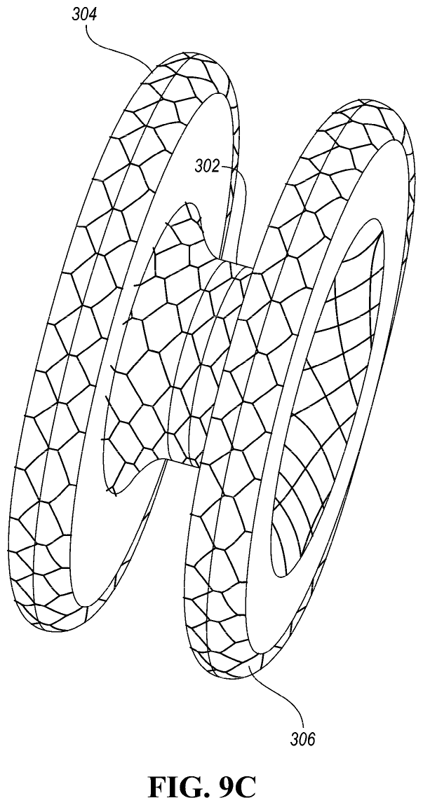

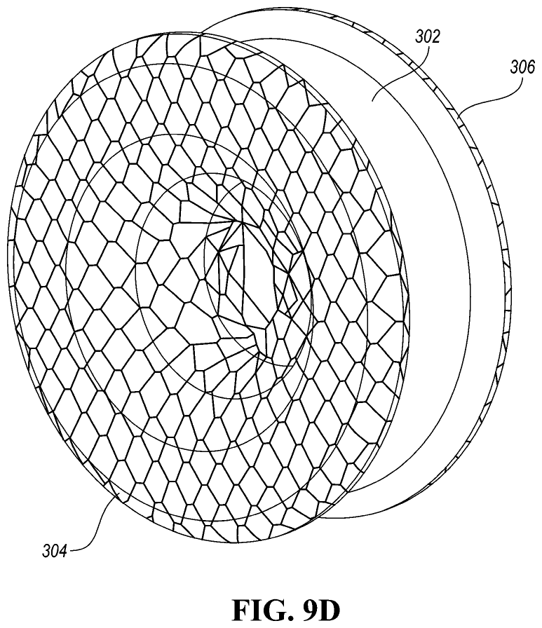

FIGS. 9A and 9B depict a schematic, side view and a schematic, perspective view, respectively, of grommet 300 including skirt 302 and first and second frame members 304 and 306 attached to skirt 302 at first and second ends 308 and 310 thereof. Skirt 302 includes first and second outer portions 312 and 314 connected to each other through middle portion 316. Skirt 302 may be formed of expanded polytetrafluoroethylene (ePTFE) or other biocompatible polymer material. First and second frame members 304 and 306 may be formed of a shape memory material such as Nitinol. First and second frame members 304 and 306 may be formed of a durable mesh material. Grommet 300 may be deployed within fenestration 318 made in-situ in stent graft 320. The same or different system may be used to form fenestration 318 and delivery and deploy grommet 300. Middle portion 316 defines channel 322 to permit blood flow therethrough.

FIG. 9C depicts a schematic, perspective view of first and second frame members 304 and 306 and skirt 302. As shown in FIG. 9C, first and second frame members 304 and 306 form a double layer stent with discs to keep first and second frame members 304 and 306 locked to stent graft 320. FIG. 9D depicts a schematic, perspective view of skirt 302. Skirt 302 is configured to prevent blood flow around first and second frame members 304 and 306.

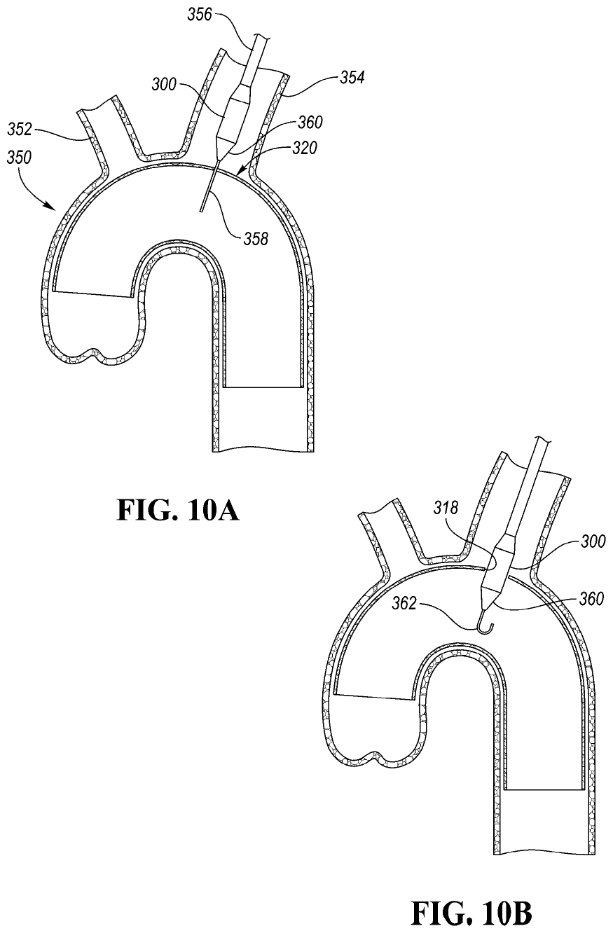

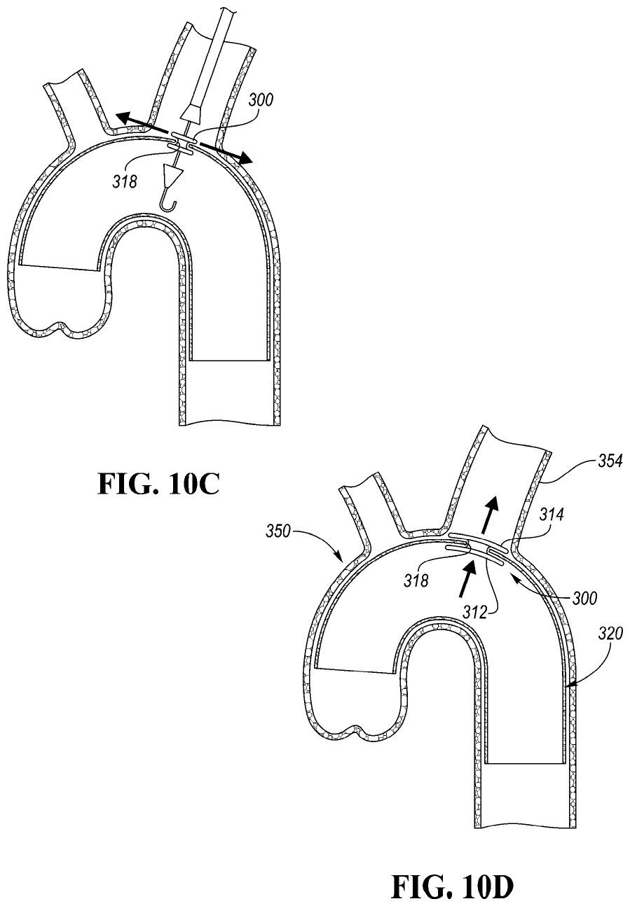

FIGS. 10A, 10B, 10C, and 10D depict schematic, cut away side views of operations for creating in-situ fenestration 318 and deploying grommet 300 within fenestration 318 according to one or more embodiments.

FIG. 10A depicts aortic arch 350 branching into left common carotid artery 352 and left subclavian artery 354. As shown in FIG. 10A, stent graft 320 is deployed within aortic arch 350, thereby covering left common carotid artery 352 and left subclavian artery 354. Delivery system 356 is tracked over guidewire 358 (e.g., straight guidewire) extending through left subclavian artery 354 and into aortic arch 350. Guidewire 358 is configured to puncture the graft material of stent graft 320 to form an initial opening. Delivery system 356 includes tapered tip 360 (e.g., dilator tip) configured to widen the initial opening in the graft material of stent graft 320 covering left subclavian artery 354. In FIG. 10A, grommet 300 is shown in a constrained position and is carried on delivery system 356. After guidewire 358 punctures the graft material of stent graft 320 to form the initial opening, it can be exchanged for guidewire 362 (shown in FIG. 10B).

As shown in FIG. 10B, tapered tip 360 is tracked along guidewire 362 and through the initial opening in the stent graft material to widen the initial opening to form fenestration 318. In FIG. 10B, grommet 300 is in the delivery position and straddles fenestration 318 during this operation for deployment as described below.

As shown in FIG. 10C, grommet 300 is deployed within fenestration 318 by increasing the diameter of grommet 300, thereby widening fenestration 318 to create a blood flow path.

As shown in FIG. 10D, delivery system 356 and guidewire 362 has been removed from the patient's vasculature (e.g., aortic arch 350 and left subclavian artery 354) through the opening defined by grommet 300. First and second outer portions 312 and 314 of grommet 300 are situated on either side of stent graft 320 and are configured to hold grommet 300 in place at fenestration 318 of stent graft 320.

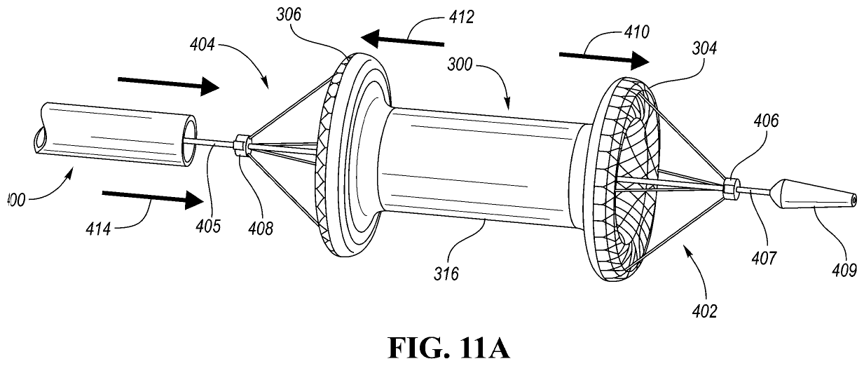

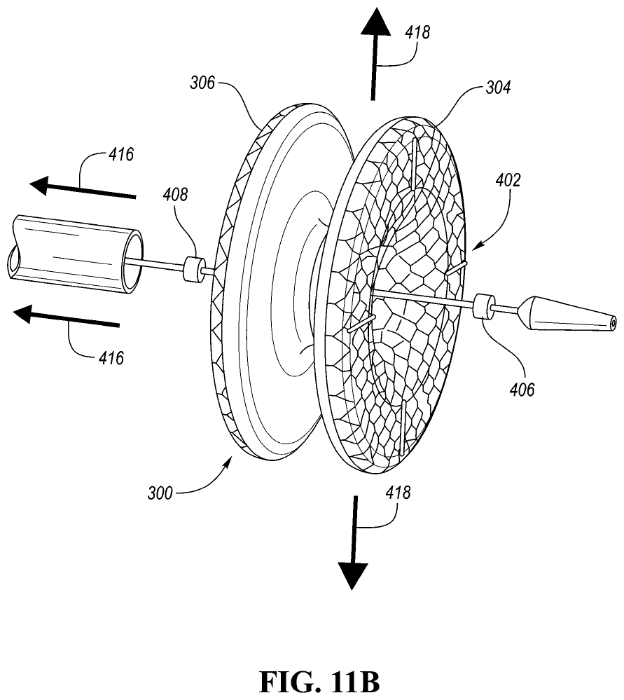

FIG. 11A depicts a schematic, perspective view of a procedure for loading grommet 300 into capsule 400 of delivery system 356. First and second tethers 402 and 404 (e.g., paddle arms) are attached at one end to first and second frame members 304 and 306, respectively. First and second tethers 402 and 404 are attached at the other end to first and second spindles 406 and 408, respectively. First and second spindles 406 and 408 are translated as shown by arrows 410 and 412, respectively, to elongate middle portion 316 of grommet 300 such that the overall profile of grommet 300 is reduced. Delivery system 356 includes separate shafts 405 and 407. In one embodiment, shaft 407 is axially fixed (e.g., stationary and/or rigid) and shaft 405 is configured for axial movement relative to shaft 407 by actuation of a handle attached to delivery system 356. The axial movement is configured to elongate or shorten grommet 300. Alternatively, both shafts may be moveable by a single actuation. Once grommet 300 has a reduced profile, capsule 400 is advance over grommet 300 as shown by arrows 414.

FIG. 11B depicts a schematic, perspective view of a procedure for deploying grommet 300 into a fenestration (e.g., fenestration 318 of FIGS. 9A and 9B). As shown by arrows 416 in FIG. 111B, capsule 400 is retracted to allow grommet 300 to radially expand, thereby widening the initial fenestration as shown by arrows 418. After grommet 300 radially expands, first and second paddle arms 402 and 404 are released from spindles 406 and 408, respectively, thereby creating free ends of first and second tethers 402 and 404. The released first and second tethers 402 and 404 are then secured (e.g., heat set) flush against first and second frame members 304 and 306, respectively.

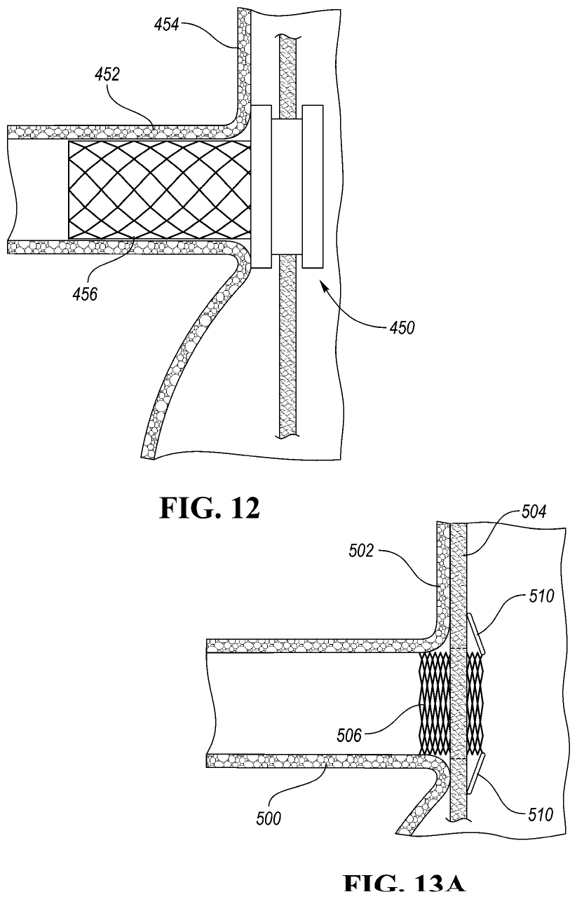

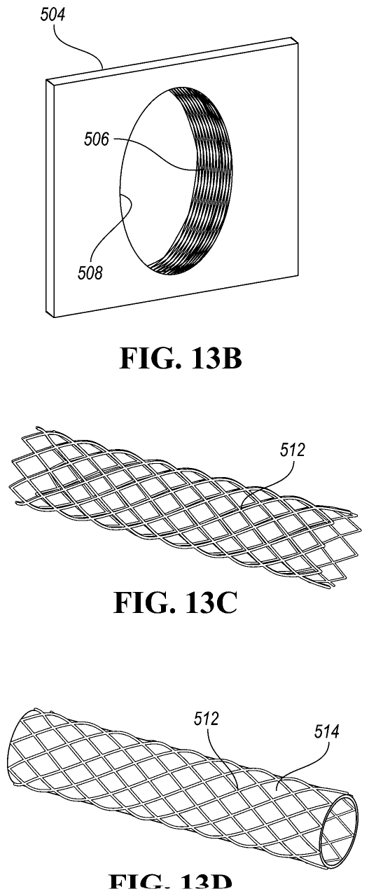

FIG. 12 depicts a schematic, perspective view of grommet 450 deployed between peripheral artery 452 and aortic artery 454. Grommet 450 may be deployed into a distal end of branching stent graft 456 with branching stent graft 456 attached to grommet 450.

One or more embodiments may have one or more of the following benefits. The grommet is configured to maintain its position within an in-situ fenestration. The grommet may resist fraying of the fabrics around the fenestration. The grommet may be manufactured in variable diameter sizes, thereby providing multiple sized grommets to vary the diameter. The grommet of one or more embodiments may provide a single device for various hole sixes. The grommet of one or more embodiments may provide adequate anchoring for branches. The grommet of one or more embodiments may provide facile viewing under Fluoro.

In one or more embodiments, a grommet configured to form an interface between a stent graft and a peripheral stent graft is disclosed. The grommet may include a tubular frame and a tubular skirt connected thereto. The grommet may include struts extending from one end thereof. The struts are configured to transition from a delivery position to a deployment position in which it is anchored to the stent graft. The grommet is configured to transition from a delivery position into a deployment position in which it is anchored within an in-situ fenestration made in the stent graft. The width of the grommet is smaller in the delivery position. The length of the grommet is longer in the delivery position. The grommet may be deployed within an in-situ fenestration to resist leakage therethrough and/or tearing of the in-situ fenestration.

FIG. 13A depicts a schematic, side view of branching artery 500 branching from aortic artery 502 with stent graft 504 deployed within aortic artery 502 and grommet 506 deployed within in-situ fenestration 508. Grommet 506 may be anchored to stent graft 504 with struts 510. Struts 510 may be formed of a shape memory material such as Nitinol. In one or more embodiments, a number (e.g., 4, 5, 6, 7, 8, 9, 10, 11, or 12) of struts 510 are circumferentially arranged around the distal end of grommet 506. Grommet 506 defines an inner channel configured to provide a blood flow path between branching artery 500 and aortic artery 502.

FIG. 13B depicts a schematic, fragmented, perspective view of stent graft 504 having fenestration 508 with grommet 506 deployed therein.

FIG. 13C depicts a schematic, perspective, isolated view of frame 512 of grommet 506. Frame 512 may be formed of a metal alloy material such as a cobalt chromium metal alloy. Frame 512 may be configured to crimp with struts 510 upon expansion of frame 512, thereby folding back the ends of frame 512.

FIG. 13D depicts a schematic, perspective view of frame 512 and skirt 514 of grommet 506. Skirt 514 may be configured to prevent blood-flow around frame 512. Frame 512 and skirt 514 may be connected to each other through stitching and/or struts 510.

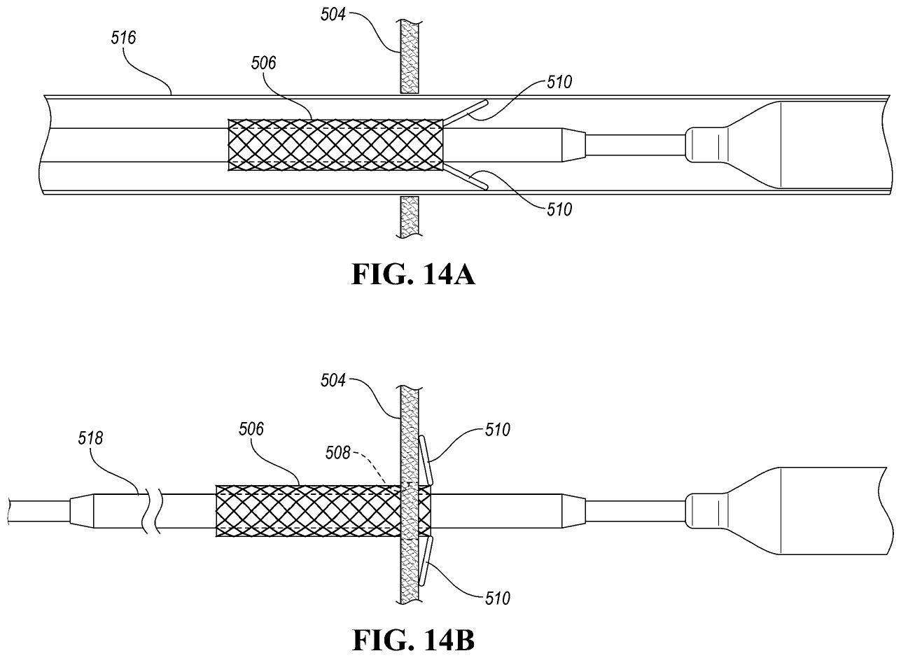

FIG. 14A depicts a schematic, side view of grommet 506 being delivered within capsule 516. As shown in FIG. 14A, struts 510 are located on a distal end of grommet 506 and are angled outward away from stent graft 504. Struts 510 are held in a delivery position within capsule 516.

FIG. 14B depicts a schematic, side view of grommet 506 during a deployment operation. Once capsule 516 is retracted, struts 510 are exposed and return to a preset, deployment position, thereby clamping to stent graft 504. At this point, balloon 518 is expanded, thereby increasing the size of in-situ fenestration 508 with grommet 506 situated therein.

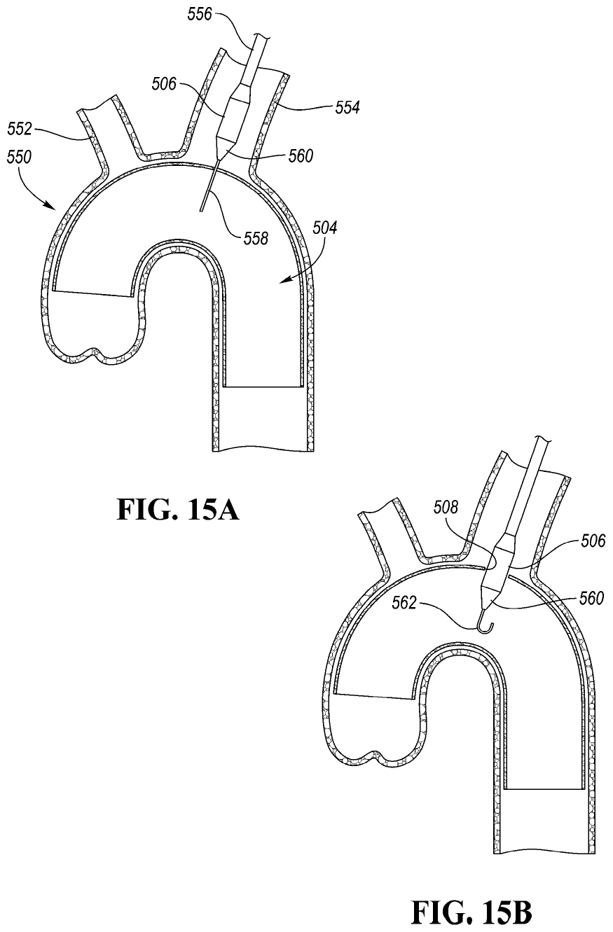

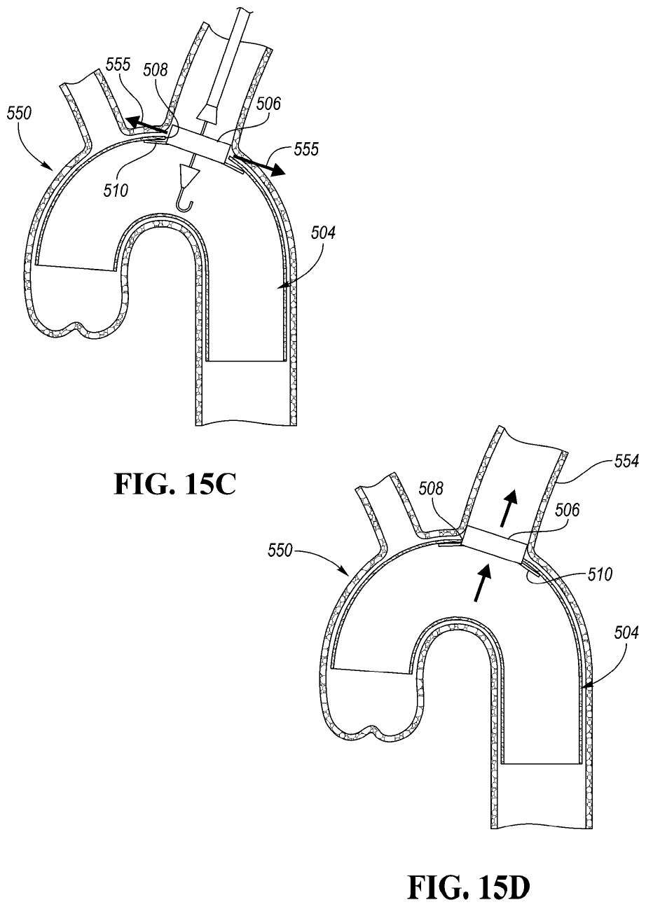

FIGS. 15A, 15B, 15C, and 15D depict schematic, cut away side views of operations for creating in-situ fenestration 508 and deploying grommet 506 within fenestration 508 according to one or more embodiments.

FIG. 15A depicts aortic arch 550 branching into left common carotid artery 552 and left subclavian artery 554. As shown in FIG. 15A, stent graft 504 is deployed within aortic arch 550, thereby covering left common carotid artery 552 and left subclavian artery 554. Delivery system 556 is tracked over guidewire 558 (e.g., straight guidewire) extending through left subclavian artery 554 and into aortic arch 550. Guidewire 558 is configured to puncture the graft material of stent graft 504 to form an initial opening. Delivery system 556 includes tapered tip 560 (e.g., dilator tip) configured to widen the initial opening in the graft material of stent graft 504 covering left subclavian artery 554. In FIG. 15A, grommet 506 is shown in a constrained position and is carried on delivery system 556. After guidewire 558 punctures the graft material of stent graft 504 to form the initial opening, it can be exchanged for guidewire 562 (shown in FIG. 10B).

As shown in FIG. 15B, tapered tip 560 is tracked along guidewire 562 and through the initial opening in the stent graft material to widen the initial opening to form fenestration 508. In FIG. 15B, grommet 506 is in the delivery position and straddles fenestration 508 during this operation for deployment as described below.

As shown in FIG. 15C, grommet 506 is deployed within fenestration 508 by increasing the diameter of grommet 506 (as shown by arrows 555) and decreasing its length, thereby widening fenestration 508 to create a blood flow path. During this step, struts 510 engage the stent graft material around the periphery of fenestration 508, thereby anchoring grommet 506 to the stent graft material.

As shown in FIG. 15D, delivery system 556 and guidewire 562 has been removed from the patient's vasculature (e.g., aortic arch 550 and left subclavian artery 554) through the opening defined by grommet 506. Grommet 506 is left behind within fenestration 508 and radial force keeps it in place with the stent graft material. Grommet 506 is anchored against left subclavian artery 554 with struts 510 anchoring grommet 506.

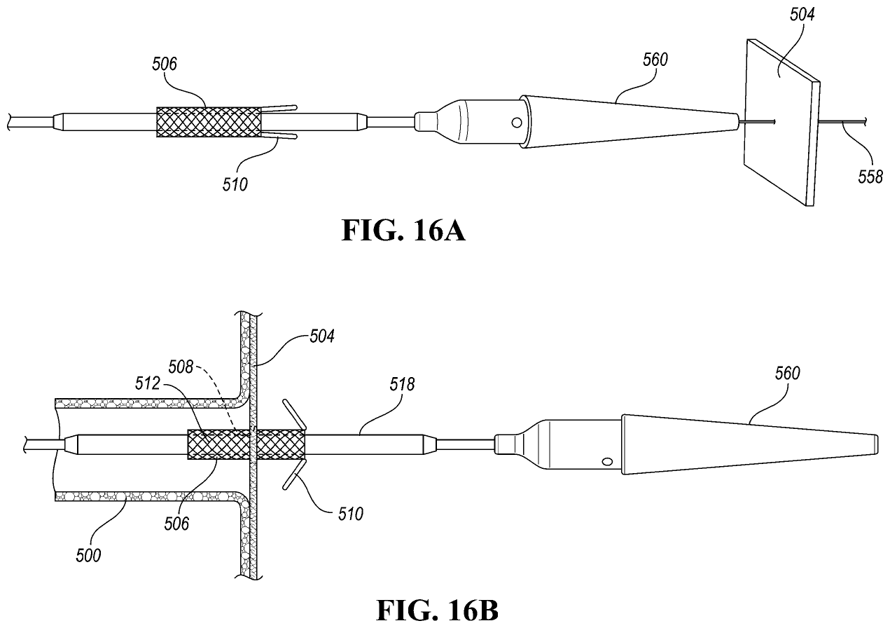

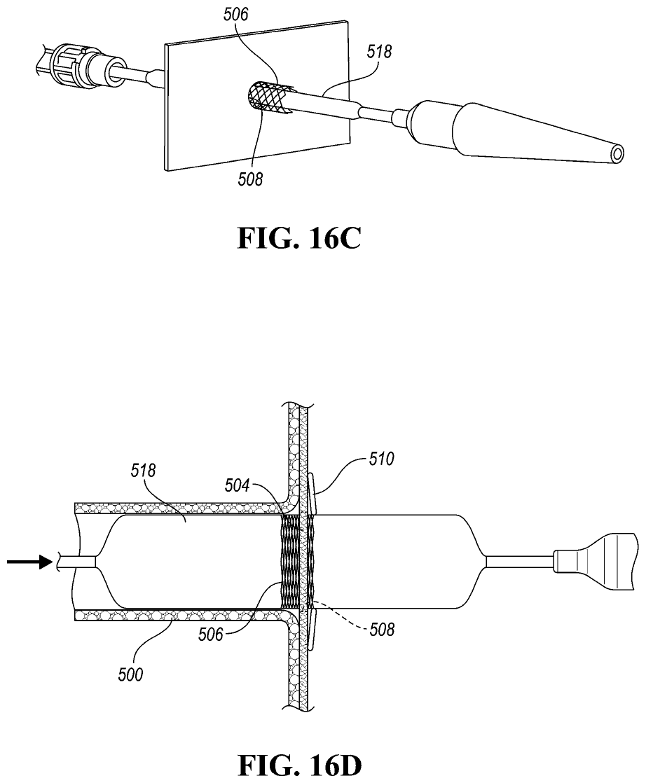

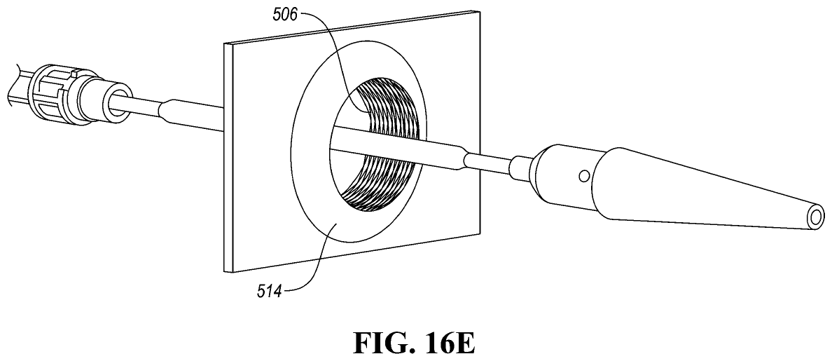

FIGS. 16A, 16B, 16C, 16D, and 16E depict schematic side and perspective views of a procedure for deploying grommet 506 into a fenestration (e.g., fenestration 508 of FIGS. 13A and 13B).

In FIG. 16A, guidewire 558 punctures the stent graft material of stent graft 504 to from an initial opening in the stent graft material. Grommet 506 and struts 510 are proximal tapered tip 560. Grommet 506 and struts 510 are shown in a delivery position. Capsule 516 constrains grommet 506 and struts 510 although capsule 516 is not shown in FIG. 16A for purposes of visualization.

In FIG. 16B, tapered tip 560 has advanced through branching artery 500 and the stent graft material of stent graft 504 to widen the initial opening to form fenestration 508. Capsule 516 has been retracted such that struts 510 have moved into their preset, deployment position. Struts 510 are attached to the distal end of frame 512 of grommet 506. Balloon 518 extends through grommet 506. As shown in FIG. 13B, balloon 518 has a longitudinal length longer than grommet 506 in its delivery position.

FIG. 16C is a schematic, perspective view of grommet 506 and balloon 518 extending through fenestration 508.

FIG. 16D is a schematic, side view of balloon 518 in an expanded, deployment position. Balloon 518 may be inflated from a deflated, delivery position into the expanded, deployment position. The inflation of balloon 518 may increase the diameter of balloon 518 by any of the following factors: 1.5, 2., 2.5, 3, 3.5, and 4. As balloon 518 expands, grommet 506 shortens, thereby both creating an anchor to branching artery 500 and an anchor to the stent graft material with struts 510. Grommet 506, in its deployed state, is configured to create a seal around fenestration 508 of stent graft 504 and branching artery 500.

FIG. 16E is a schematic, perspective view of grommet 506 showing skirt 514 extending around and covering struts 510 (not shown).

In one or more embodiments, a device to form an in-situ fenestration and deploy a grommet therein is disclosed. The device carries a grommet in a crimped state and includes a balloon for expanding the grommet into an expanded state. The device includes an adhesive channel configured to dispense adhesive into a channel of the grommet after the balloon expands the grommet into the expanded state.

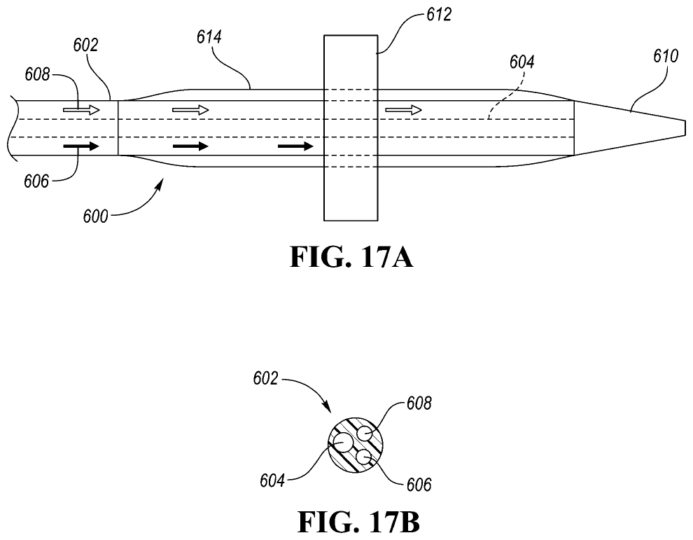

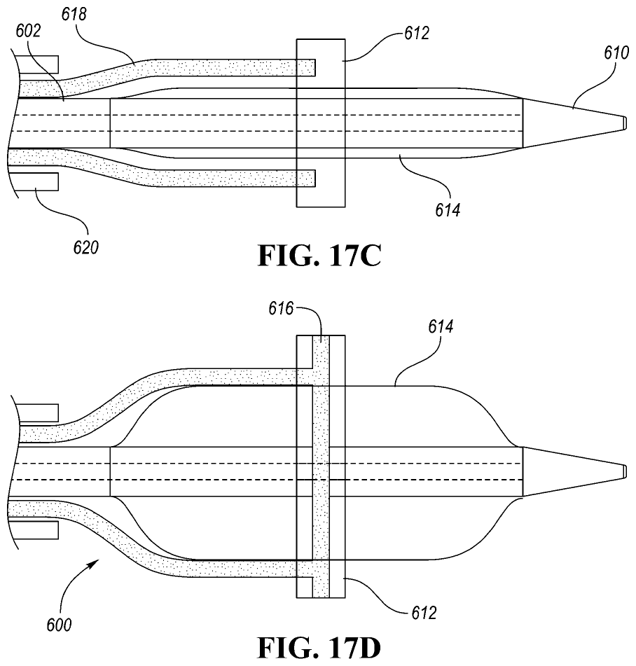

FIG. 17A depicts a schematic, side view of delivery catheter 600 including inner shaft 602 according to one embodiment. FIG. 17B depicts a schematic, cross-sectional view of inner shaft 602 of delivery catheter 600. As shown in FIG. 17B, steerable inner lumen 604, curing light source 606, and inflation media 608 are housed within inner shaft 602. Tapered tip 610 is connected to the distal end of shaft 602 and is configured to pierce through stent graft material. Tapered tip 610 and steerable inner lumen 604 are connected to each other such that steerable inner lumen 604 is configured to steer tapered tip 610 through a patient's vasculature. Grommet 612 is crimped to balloon 614 to provide a smooth transition between proximal tapered tip 610 and grommet 612. Delivery catheter 600 is configured to create an initial hole for an in-situ fenestration, position grommet 612, and expand grommet 612. There are at least two mechanisms to affix grommet 612 at the fenestration site. Grommet 612 includes central channel 616 configured to receive an adhesive (e.g., glue) delivered through adhesive channel 618. In one or more embodiments, the adhesive has a viscosity low enough to be delivered through a tube about 1 millimeter in diameter. Once the adhesive fills central channel 616, the adhesive is crosslinked using curing light. In one embodiment, the adhesive may be a hemostatic hydrogel, such as the hemostatic hydrogel referenced in Hong, Y., Zhou, F., Hua, Y. et al., A strongly adhesive hemostatic hydrogel for the repair of arterial and heart bleeds. Nat Commun 10, 2060 (2019), which is herein incorporated by reference in its entirety. The curing light for this adhesive may have a frequency of 365 nm. Grommet 612 may be formed of a translucent material to permit curing of the adhesive. Grommet 612 may be formed of a silicone material (e.g., an injection molded, low durometer clear silicone).

FIG. 17C depicts a schematic, side view of adhesive channel 618 situated between inner shaft 602 and outer shaft 620. FIG. 17C depicts delivery catheter 600 in a first deployment position where adhesive channel 618 extends from outer shaft 620 and into grommet 612. Outer shaft 620 may have a size of less than or equal to 22 French (e.g., 18, 19, 20, 21, or 22 French). Grommet 612 may be delivered transfemorally, however, access may also be provided via radial or carotid access.

FIG. 17D depicts a schematic, side view of delivery catheter 600 in a second deployment position. In the second deployment position, balloon 614 is inflated into an expanded position, thereby expanding the diameter (e.g., by 1.5, 1.6, 1.7, 1.8, 1.9, 2.0, 2.1, 2.2, 2.3, 2.4, and 2.5 times) of grommet 612. As shown in FIG. 17D, the adhesive fills central channel 616 of grommet 612.

In one or more embodiments, the graft material removed to form the in-situ fenestration is secured to the expanded grommet rather than being removed from the patient. The grommet may be sized to create an in-situ fenestration in front of the left subclavian or left carotid arteries. The grommet delivery system may resist damage to the stents or sutures of the stent graft during creation of the in-situ fenestration. The in-situ fenestration dilation and grommet deployment may be part of the same procedure. The grommet may be configured to provide support to the stent graft during dilation. The adhesive may be configured to seal excess graft material in the grommet to resist fraying. The adhesive may also be configured to maintain the grommet in an expanded position after deployment. Ultraviolet curing permits the adhesive to cure/set in seconds (e.g., 2, 3, 4, or 5 seconds). The curing light may be delivered through a delivery catheter through a tri-lumen shaft.

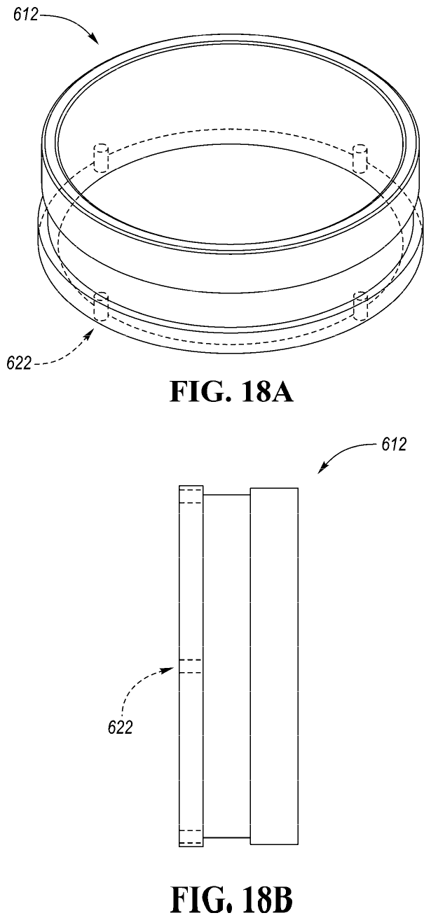

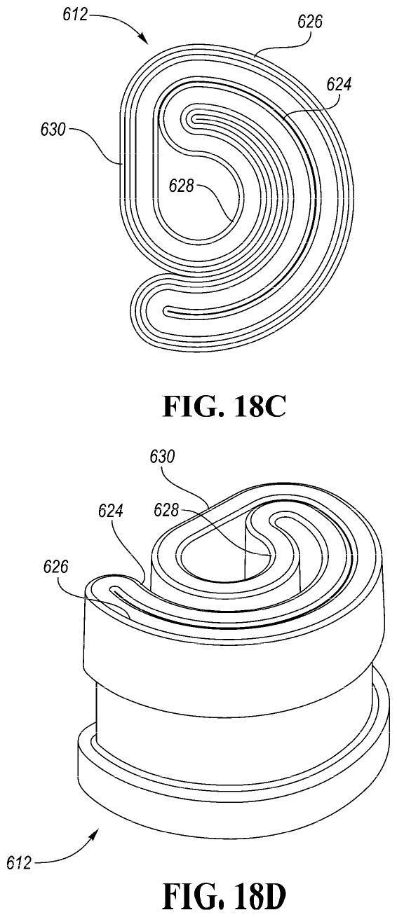

FIG. 18A depicts a schematic, perspective view of grommet 612 in an expanded state. FIG. 18B depicts a schematic side view of grommet 612 in the expanded state. In one or more embodiments, grommet 612 has a recess or channel in a middle region thereof where it can seat in the peripheral edge of a stent graft fenestration with larger diameter portions on either side of the middle region to anchor grommet 612. The adhesive is configured to help to lock grommet 612 to this location in addition to mitigating fraying and other benefits identified herein.

As shown in FIGS. 18A and 18B, grommet 612 includes adhesive ports 622. While 4 adhesive ports are shown, there may be less or more adhesive ports (e.g., 2, 3, 5, 6, 7, or 8) depending on the embodiment. In the expanded state, the diameter of grommet 612 may be any of the following values or in a range of any two of the following values: 8, 9, 10, 11, 12, 13, and 14 millimeters. In the expanded state, the circumference/perimeter of grommet 612 may be any of the following values or in a range of any two of the following values: 25, 26, 27, 28, 29, 30, 31, 32, 33, 34, 35, 36, 37, 38, 39, 40, 41, 42, 43, and 44 millimeters. In the expanded state, the width of grommet 612 may be any of the following values or in a range of any two of the following values: 2.5, 2.6, 2.7, 2.8, 2.9, 3.0, 3.1, 3.2, 3.3, 3.4, and 3.5 millimeters. Specific grommets may be formed for specific sizes. In one specific example, a 2 millimeter balloon expands to 10 millimeters in outer diameter, thereby expanding grommet 612 into a 31.4 millimeter circumference/perimeter.

FIG. 18C depicts a schematic, plan view of grommet 612 in a crimped state. FIG. 18D depicts a schematic, perspective view of grommet 612 in the crimped state. The inner perimeter of grommet 612 in the crimped state may match (e.g., equal) the inner diameter of grommet 612 in the expanded state. The radius of grommet 612 in the crimped state may be any of the following values or in a range of any two of the following values: 4.0, 4.5, 5.0, 5.5, 6.0, 6.5, and 7.0 millimeters. As shown in FIGS. 18C and 18D, grommet 612 in the crimped state forms an inner channel configured to permit the diameter of balloon 614. The diameter of balloon 614 may be any of the following values or in a range of any two of the following values: 2.0, 2.1, 2.2, 2.3, 2.4, and 2.5, 3.0, 4.0, 5.0, 6.0, and 7.0 millimeters. In one specific example, a 2 millimeter balloon expands to 10 millimeters in outer diameter, thereby expanding grommet 612 into a 31.4 millimeter circumference/perimeter. Grommet 612 in the crimped state includes first and second major radiuses portions 624 and 626, minor radiused portion 628, and straight portion 630. Grommet 612 may be in the crimped state during delivery through the patient's vasculature.

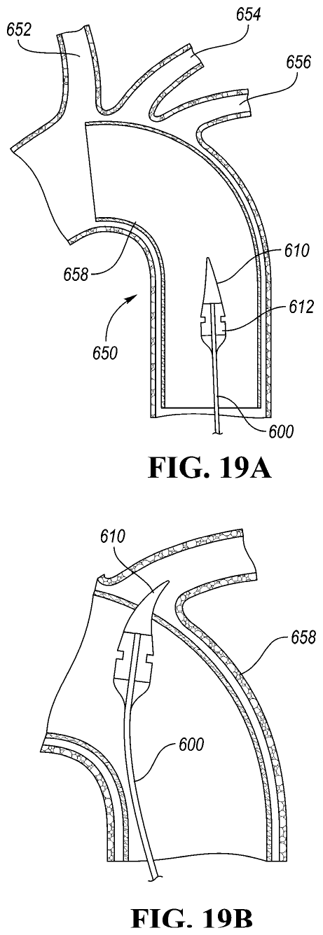

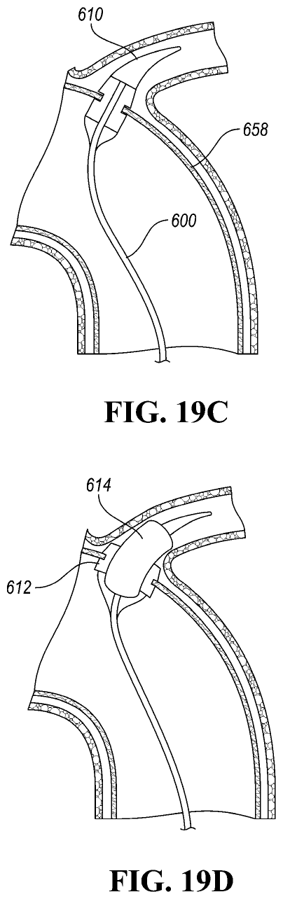

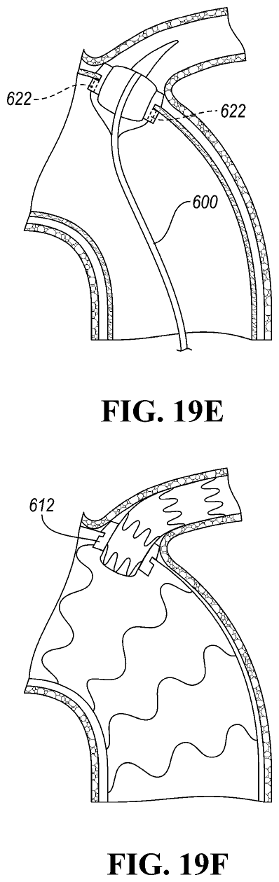

FIGS. 19A, 19B, 19C, 19D, 19E, and 19F depict schematic, side views of aortic arch 650 branching into brachiocephalic artery 652, left common carotid artery 654, and left subclavian artery 656 with stent graft 658 deployed in the aortic arch 650 and delivery catheter 600 tracking transfemorally to stent graft 658. Delivery catheter 600 is configured to create an in-situ fenestration in stent graft 658 and deploy grommet 660 therein. Grommet 612 is in a crimped state as shown in FIG. 19A. Delivery catheter 600 includes tapered tip 610 at the distal end thereof. As shown in FIG. 19B, tapered tip 619 is configured to puncture a hole in the stent graft material of stent graft 658. In one or more embodiments, a needle may be advanced through the stent graft material before tapered tip 610 is advanced therethrough. Delivery catheter 600 may be steerable to allow navigation to correct vessel.

As shown in FIG. 19C, delivery catheter 600 advances such that grommet 612 in the crimped state crosses the graft material of stent graft 658. In one or more embodiments, grommet 612 may be centered radially within the in-situ fenestration formed in stent graft 658. The diameters of grommet 612 and tapered tip 610 may be matched so that a smooth transition from tapered tip 610 occurs as tapered tip 610 advances.

As shown in FIG. 19D, balloon 614 is inflated to expand grommet 612 into the expanded state. As shown in FIG. 19E, adhesive (e.g., glue) is dispensed through adhesive ports 622. Ultraviolet light may shine through the central lumen of catheter device 600 and through balloon 614 to cure the adhesive. The material of grommet 612 may be clear so that curing light cures the adhesive. As shown in FIG. 19F, grommet 612 is in its fully deployed position within the in-situ fenestration and balloon 614 has been deflated and catheter device 600 has been removed.

The detailed description set forth herein includes several embodiments where each of the embodiments include several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any systems and methods for locating a branch ostium of a branch vessel disclosed herein may be used in conjunction with any disclosed embodiments. Similarly, any systems, methods, or energy types for creating a fenestration (e.g., heat, laser, vibration, RF energy, blades/mechanical cutting) may be used in any disclosed embodiments. In any of the embodiments disclosed herein, following the creation of a fenestration the fenestration may be reinforced or strengthened by placing a stent or grommet like device in the fenestration. After a fenestration is created (and optionally reinforced), a branch stent graft may be tracked and deployed within the fenestration using a separate delivery system. The branch stent graft may extend within the fenestration and at least partially within a main lumen of the fenestrated stent graft and into branch artery (e.g., renal artery, celiac, SMA, BCA, LCC, LSA, etc.). The systems, methods, and devices disclosed herein may be used to make multiple fenestrations in a single stent graft, which thereafter each receive a branch stent graft. Vacuum aspiration may be used in conjunction with any of the devices or methods described herein to remove potential emboli (e.g., gas or pieces of graft material) from the fenestration site. Vacuum aspiration may be applied through a lumen of the fenestration system or may be provided by a standalone aspiration catheter.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An endovascular grommet comprising:
a central member including a central portion and first and second portions extending from the central portion;
an inner ring connected to the central member;
a first outer ring connected to the central member;
a second outer ring connected to the central member;
a first coil secured to the first portion of the central member; and
a second coil secured to the second portion of the central member,
the grommet configured for deployment within an in-situ fenestration.

2. The endovascular grommet of claim 1, wherein the first outer ring is connected to the first portion of the central member and the second outer ring is connected to the second portion of the central member.

3. The endovascular grommet of claim 1, wherein the first portion of the central portion includes first inner and outer peripheral ends, the first outer peripheral end terminates in the first outer ring.

4. The endovascular grommet of claim 1, wherein the first coil extends along a first circumferential axis and the second coil extends along a second circumferential axis.

5. The endovascular grommet of claim 4, wherein the first outer ring extends within the first coil.

6. The endovascular grommet of claim 4, wherein the first outer ring extends within the first coil offset from the first circumferential axis.

7. The endovascular grommet of claim 1, wherein the central portion of the central member is connected to the inner ring.

8. The endovascular grommet of claim 1, wherein the first and second portions of the central member have opposing conical frustrum shapes.

9. The endovascular grommet of claim 1, wherein the central member includes a mesh structure covered with a polymeric material or a fabric material.

10. The endovascular grommet of claim 1, wherein the first and second portions of the central member have first and second radial cross sections forming a cone shape.

11. An endovascular stent graft system comprising:
a main stent graft defining a fenestration and including stent graft material;
a peripheral stent graft;
a grommet forming an interface between the main stent graft and the peripheral stent graft to resist leakage through the fenestration, the grommet including:
a central member including a central portion and first and second portions extending from the central portion;
an inner ring connected to the central member;
a first outer ring connected to the central member;
a second outer ring connected to the central member;
a first coil secured to the first portion of the central member; and
second coil secured to the second portion of the central member,
the peripheral stent graft extending through the main stent graft and the grommet.

12. The endovascular stent graft system of claim 11, wherein the first and second coils exert a pinching force to pinch the stent graft material of the main stent graft.

13. The endovascular stent graft system of claim 11, wherein the first and second coils mesh with each other to pinch the stent graft material of the main stent graft.

14. The endovascular stent graft system of claim 11, wherein the first and second coils exert an outward force on the grommet, the inner ring exerts an inward force balancing the outward force.

15. The endovascular stent graft system of claim 11, wherein the peripheral stent graft includes inner and outer surfaces, the inner ring of the grommet contacts the outer surface of the peripheral stent graft.

16. An endovascular grommet deployment system comprising:
a capsule; and
a grommet having a constrained position within the capsule and a deployed position outside of the capsule, the grommet including a central member, an inner ring connected to the central member, first and second outer rings connected to the central member, and first and second coils secured to the central member,
the grommet configured to straddle stent graft material defining a fenestration when the grommet is in the deployed position.

17. The endovascular grommet deployment system of claim 16 further comprising retainer located between the capsule and the grommet and configured to retain the grommet when the grommet is in the constrained position.

18. The endovascular grommet deployment system of claim 17, wherein the retainer includes at least two prongs.

19. The endovascular grommet deployment system of claim 18, wherein the at least two prongs include a first prong and a second prong, the first prong includes a first inwardly curved channel, the second prong includes a second inwardly curved channel, and the first and second inwardly curved channels configured to retain the grommet in the constrained position.

20. The endovascular grommet deployment system of claim 16, wherein the grommet defines an opening in the deployed position configured to receive a peripheral stent graft.
